(19) United States
(12) Patent Application Publication
(10) Pub. No.: US 2007/0112250 A1
(43) Pub. Date: May 17, 2007

(54) Insertion support tool for medical instrument and insertion method for medical instrument

(76) Inventors: Yasuhito Kura, Hachioji-shi (JP); Katsutaka Adachi, Hino-shi (JP); Shigeki Matsuo, Tokyo (JP); Tsutomu Ishiguro, Hino-shi (JP); Ryuichi Toyama, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 11/288,453

(22) Filed: Nov. 16, 2005

(51) Int. Cl. A61B 1/00

(52) U.S. Cl. 600/114; 600/156

(57) ABSTRACT

An insertion support tool which is detachably attached to a large-intestine endoscope including an elongated inserting portion which is inserted into a lumen is provided, and an external sheath which is extendable and contractible in an inserting direction of the inserting portion and a cover member which is arranged outside the external sheath and held in a state where it covers the external sheath when the external sheath is contracted are provided to this insertion support tool.

### BACKGROUND OF THE INVENTION

1. Field of the Invention

The present invention relates to an insertion support tool for a medical instrument and an insertion method for a medical instrument, which support an operation of inserting, e.g., a large-intestine microscope into a body.

2. Description of the Related Art

At the time of an operation of inserting a large-intestine endoscope into a patient's body, an operation of inserting an end portion of the endoscope into an anus and further pushing it into a large intestine is carried out. In general, the large intestine has many bent parts in the middle of a long tract and hence has a complicated shape. Therefore, the operation of inserting the endoscope into the large intestine is a relatively difficult operation.

As a technology which supports such an operation of inserting the endoscope into the large intestine, International Publication No. 02/19886 discloses an endoscope. In this publication, a bag-shaped exterior sheath is attached in a folded state on an outer side of an elongated inserting portion which is inserted into a body. Further, at the time of an operation of inserting the endoscope, a gas is injected into a bag of the exterior sheath. As a result, the exterior sheath is extended. Furthermore, the endoscope is led into the large intestine by this extending operation of the exterior sheath. Since this exterior sheath is extended by elastically deforming this folded portion, it is desirable for the exterior sheath to have flexibility and to be readily extended.

It is normal that the anus of a patient is closed by a sphincter muscle. Therefore, when inserting the endoscope, insertion is carried out while spreading the sphincter muscle of the anus.

### BRIEF SUMMARY OF THE INVENTION

It is an object of the present invention to provide an insertion support tool for a medical instrument and an insertion method for a medical instrument which have been improved in quality.

According to a first aspect of the present invention, there is provided an insertion support tool for a medical instrument, comprising: a transforming portion which is expandable and contractible along an inserting direction of an inserting portion of the medical instrument having flexibility into a lumen; and a cylindrical cover member which is held to cover the transforming portion when the transforming portion is contracted.

The cover member may be formed of a material which can prevent an external force applied to a surface of this cover member from acting on the transforming portion.

The transforming portion may have a bag-shaped sheath which is arranged to cover at least a part of a side surface of the inserting portion in the inserting direction and which is extendable in the inserting direction of the inserting portion by supply of a fluid.

The insertion support tool may further have a holding member which maintains a state where the cover member covers the transforming portion when the transforming portion is contracted.

The holding member may have a packing material which is used to pack the insertion support tool.

The insertion support tool may further comprise a protection member including a fixing portion at which at least a part of the insertion support tool is fixed, and the protection member may be inserted into and fixed in the lumen prior to insertion of the medical instrument into the lumen.

According to a second aspect of the present invention, there is provided an insertion support tool for a medical instrument, which is detachably attached to the medical instrument including a shaft-shaped inserting portion having flexibility which is inserted into a lumen, comprising: a bag-shaped transforming portion which is arranged outside the insertion portion and which is extendable in an inserting direction of the inserting portion by supply of a fluid; a base unit having a first transforming portion fixing portion at which a base end portion of the transforming portion is fixed; an end unit having a second transforming portion fixing portion at which an end portion of the transforming portion is fixed; and a cylindrical cover member which is arranged outside the transforming portion, whose base end portion is fixed to the based unit, and which is held to cover the transforming portion when the transforming portion is contracted.

The transforming portion may have an outer sheath having a folded first bellows portion and an inner sheath which is arranged in the outer sheath and has a folded second bellows portion, and the base unit may have a fluid path through which a working fluid is supplied between the outer sheath and the inner sheath.

The end unit may have an engagement member which is releasably engaged with an end portion of the cover member and maintains the state where the cover member covers the transforming portion when the transforming portion is contracted.

The engagement member may have a tape which locks a part between the end unit and the cover member.

The engagement member may have a fixed thread having one end fixed to the end unit and the other end fixed to the base unit.

The engagement member may have a concavoconvex fitting portion which is formed at a joint portion of the end unit and the cover member.

According to a third aspect of the present invention, there is provided an insertion method for a medical instrument, comprising: a step of attaching an insertion support tool to the medical instrument including a shaft-shaped inserting portion which is inserted into a lumen; a first insertion step of inserting the insertion support tool together with the medical instrument into the lumen while maintaining a state where a cylindrical cover member covers a transforming portion which is extendable and contractible in an inserting direction of the insertion portion after attaching the insertion support tool; and a second insertion step of fixing a base end portion of the insertion support tool in an anus part, then extending the transforming portion in the inserting direction of the insertion portion to protrude the medical instrument from the cover member so that the medical instrument is moved forward in the inserting direction of the inserting portion.

According to a fourth aspect of the present invention, there is provided an insertion support tool for a medical instrument, having: a first extension unit which is extendable and contractible in an inserting direction of an inserting portion of the medical instrument having flexibility into a lumen; a second extension unit which is extendable and contractible in the inserting direction separately from the first extension unit; and a coupling member which couples the first extension unit with the second extension unit.

The second extension unit may be arranged at an end portion of the first extension unit.

The first extension unit may comprise: a bag-shaped first transforming portion which is arranged to cover at least a part of a side surface of the inserting portion in the inserting direction and which is extendable in the inserting direction by supply of a fluid; a base unit having a first transforming portion fixing portion to which a base end portion of the first transforming portion is fixed; an end unit having a second transforming portion fixing portion to which an end portion of the first transforming portion is fixed; and a cylindrical cover member whose end portion is fixed to the end unit and which is maintained to cover the first transforming portion when the first transforming portion is contracted, the second extension unit may have: a bag-shaped second transforming portion which is arranged to cover at least a part of the side surface of the inserting portion in the inserting direction and which is extendable in the inserting direction by supply of a fluid; a cylindrical rear side frame member coupled with a base end portion of this second transforming portion; and an end side frame member coupled with an end portion of the second transforming portion, and the coupling member may have a coupling tube whose end is fixed to the rear side frame member and whose base end is fixed to the end unit.

The first transforming portion may have a first outer sheath having a folded first bellows portion and a first inner sheath which is arranged on the inner side of the first outer sheath and has a folded second bellows portion, the base unit may have a first fluid path through which a working fluid is supplied between the first outer sheath and the first inner sheath, the second transforming portion may have a second outer sheath having a folded third bellows sheath portion and a second inner sheath which is arranged on the inner side of this second outer sheath and has a folded fourth bellows portion, and the rear side frame member may have a second fluid path through which a working fluid is supplied between the second outer sheath and the second inner sheath.

The insertion support tool may have an indicating portion which indicates identification data of the medical instrument to be combined.

According to a fifth aspect of the present invention, there is provided an insertion method for a medical instrument, comprising: a step of attaching an insertion support tool to the medical instrument including a shaft-shaped inserting portion having flexibility which is inserted into a lumen; a first insertion step of inserting the insertion support tool together with the medical instrument into the lumen after attaching the insertion support tool; a second insertion step of fixing a base end portion of the insertion support tool in an anus part, and then extending the first transforming portion in an inserting direction of the inserting portion to move forward the medical instrument in the inserting direction of the inserting portion; and a third insertion step of extending the second transforming portion in the inserting direction of the inserting portion to move forward the medical instrument in the inserting direction of the inserting portion independently of the forward movement operation of the medical instrument by the first transforming portion.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a vertical cross-sectional view showing an outline configuration of an insertion support tool for a large-intestine endoscope according to a first embodiment of the present invention;

FIG. 2 is a vertical cross-sectional view showing a state in which the endoscope is assembled with respect to the insertion support tool according to the first embodiment;

FIG. 3A is a schematic block diagram showing a state before an assembled unit in which the endoscope is assembled with respect to the insertion support tool according to the first embodiment is inserted into an anus;

FIG. 3B is a schematic block diagram showing a state in which the assembled unit is inserted into the anus;

FIG. 4 is a vertical cross-sectional view showing a state in which the assembled unit including the insertion support tool according to the first embodiment and the endoscope is extended in a large intestine;

FIG. 5 is an explanatory view illustrating a state of coupling an air supply tube with an air supply mouthpiece of a base unit of the insertion support tool according to the first embodiment;

FIG. 6 is a schematic block diagram showing a use state of a support member of the endoscope assembled with respect to the insertion support tool according to the first embodiment;

FIG. 7A is a perspective view of a primary part showing an example of the support member of the endoscope;

FIG. 7B is a perspective view of a primary part showing a modification of the support member of the endoscope;

FIG. 8 is a vertical cross-sectional view showing an outline configuration of an insertion support tool for a large-intestine endoscope according to a second embodiment of the present invention;

FIG. 9 is a schematic block diagram showing a state where an assembled unit in which the endoscope is assembled with respect to the insertion support tool according to the second embodiment is inserted into an anus;

FIG. 10 is a vertical cross-sectional view showing a primary part configuration of an insertion support tool according to a third embodiment of the present invention;

FIG. 11 is an explanatory view illustrating a primary part configuration of an insertion support tool according to a fourth embodiment of the present invention;

FIG. 12 is a vertical cross-sectional view showing a primary part configuration of an endoscope insertion apparatus according to a fifth embodiment of the present invention;

FIG. 13 is a side view showing a primary part configuration of an insertion support tool according to the fifth embodiment;

FIG. 14 is an explanatory view illustrating a primary part configuration of an insertion support tool according to a sixth embodiment of the present invention;

FIG. 15 is a side view showing an unsealing portion of a packing bag of the insertion support tool according to the sixth embodiment;

FIG. 16 is an explanatory view illustrating an assembling operation of assembling an endoscope with respect to the insertion support tool from the unsealing portion of the packing bag of the insertion support tool according to the sixth embodiment;

FIG. 17 is an explanatory view illustrating a first modification of the packing bag of the insertion support tool according to the sixth embodiment;

FIG. 18 is a plan view showing a second modification of the packing bag of the insertion support tool according to the sixth embodiment;

FIG. 19 is a plan view showing a third modification of the packing bag of the insertion support tool according to the sixth embodiment;

FIG. 20A is a perspective view showing a fourth modification of the packing bag of the insertion support tool according to the sixth embodiment in a state where the insertion support tool is enclosed in the packing bag;

FIG. 20B is an explanatory view illustrating an operation of taking out the insertion support tool from the packing bag of the fourth modification;

FIG. 21A is a perspective view showing a fifth modification of the packing bag of the insertion support tool according to the sixth embodiment in a state where the insertion support tool is enclosed in the packing bag;

FIG. 21B is an explanatory view illustrating an operation of taking out the insertion support tool from the packing bag of the fifth modification;

FIG. 22 is a vertical cross-sectional view of a primary part showing an insertion support tool according to a seventh embodiment of the present invention;

FIG. 23 is a front view showing an endoscope fixing portion of the insertion support tool according to the seventh embodiment;

FIG. 24 is a perspective view showing the endoscope fixing portion of the insertion support tool according to the seventh embodiment;

FIG. 25 is a perspective view showing an end portion of an inserting portion of an endoscope which is assembled with respect to the insertion support tool according to the seventh embodiment;

FIG. 26 is a perspective view showing a base unit of the insertion support tool according to the seventh embodiment;

FIG. 27A is a front view showing a pressed state of an operation button of an endoscope pressing portion in the base unit of the insertion support tool according to the seventh embodiment;

FIG. 27B is a vertical cross-sectional view of a primary part showing the pressed state of the operation button according to the seventh embodiment;

FIG. 28 is a vertical cross-sectional view of a primary part showing a first modification of the endoscope pressing portion of the insertion support tool according to the seventh embodiment;

FIG. 29 is a vertical cross-sectional view of a primary part showing a pressing released state of the operation button in the first modification of the insertion support tool according to the seventh embodiment;

FIG. 30A is a lateral cross-sectional view of a primary part of a second modification of the endoscope pressing portion of the insertion support tool according to the seventh embodiment;

FIG. 30B is a lateral cross-sectional view of a primary part showing a third modification of the endoscope pressing portion;

FIG. 31A is a vertical cross-sectional view of a primary part showing a fixing released state of an endoscope pressing portion according to an eighth embodiment of the present invention;

FIG. 31B is a vertical cross-sectional view of a primary part showing a fixing state of an endoscope by the endoscope pressing portion according to the eighth embodiment;

FIG. 32 is a vertical cross-sectional view of a primary part showing an endoscope pressing portion of an insertion support tool according to a ninth embodiment of the present invention;

FIG. 33 is a vertical cross-sectional view of a primary part showing an endoscope pressing portion of an insertion support tool according to a 10th embodiment of the present invention;

FIG. 34 is a lateral cross-sectional view of a primary part showing an endoscope pressing portion of an insertion support tool according to an 11th embodiment of the present invention;

FIG. 35A is a perspective view of a primary part showing a state where an adhesive tape is attached on a flange portion of an insertion support tool according to a 12th embodiment of the present invention;

FIG. 35B is a vertical cross-sectional view of a primary part showing the flange portion of the insertion support tool according to the 12th embodiment;

FIG. 35C is a schematic block diagram of a primary part showing a state where the flange portion of the insertion support tool according to the 12th embodiment is fixed to a patient;

FIG. 35D is an explanatory view illustrating an operation of inserting the insertion support tool according to the 12th embodiment into the patient;

FIG. 35E is an explanatory view illustrating a curved state of the insertion support tool inserted into the patient according to the 12th embodiment;

FIG. 36A is a vertical cross-sectional view of a peripheral part of the flange portion, showing a modification of the flange portion of the insertion support tool according to the 12th embodiment;

FIG. 36B is a perspective view showing the flange portion of the insertion support tool according to the same modification;

FIG. 37 is a vertical cross-sectional view of a primary part showing an insertion support tool according to a 13th embodiment of the present invention;

FIG. 38A is a schematic block diagram of a primary part showing a state where an insertion support tool according to a 14th embodiment of the present invention is fixed to a patient;

FIG. 38B is a schematic block diagram of a primary part showing a state where a fixing belt is disposed to an attachment groove of the insertion support tool according to the 14th embodiment;

FIG. 39 is a vertical cross-sectional view of a primary part showing an insertion support tool according to a 15th embodiment of the present invention;

FIG. 40 is a side view showing the insertion support tool according to the 15th embodiment;

FIG. 41A is a view showing a state where a treatment instrument in an observation image of an endoscope inserted into a body by the insertion support tool according to the 15th embodiment appears from a lower side of a screen;

FIG. 41B is a view showing a state where the treatment instrument appears from an upper side of the screen;

FIG. 41C is a view showing a state where the treatment instrument appears from a left side of the screen;

FIG. 42 is a plan view of a primary part showing an insertion support tool according to a 16th embodiment of the present invention;

FIG. 43 is a plan view of a primary part showing an insertion support tool according to a 17th embodiment of the present invention;

FIG. 44 is a plan view of a primary part showing an insertion support tool according to an 18th embodiment of the present invention;

FIG. 45 is a plan view of a primary part showing an insertion support tool according to a 19th embodiment of the present invention;

FIG. 46A is a side view showing a state where the insertion support tool is twisted at the time of an operation of the insertion support tool according to the 19th embodiment;

FIG. 46B is a vertical cross-sectional view of FIG. 46A;

FIG. 47 is a plan view of a primary part showing an insertion support tool according to a 20th embodiment of the present invention;

FIG. 48 is a plan view of a primary part showing an insertion support tool according to a 21st embodiment of the present invention;

FIG. 49 is a schematic block diagram of an insertion support tool of a large-intestine endoscope according to a 22nd embodiment of the present invention;

FIG. 50 is a vertical cross-sectional view showing an end side extension unit of the insertion support tool according to the 22nd embodiment;

FIG. 51A is an explanatory view illustrating a state where an end portion of the large-intestine endoscope has reached a position of a sharp intestinal canal bent part at the time of an operation of inserting the large-intestine endoscope into the large intestine by the insertion support tool according to the 22nd embodiment;

FIG. 51B is an explanatory view illustrating a state where a curving portion of the large-intestine endoscope lodges in the sharp intestinal canal bent part and cannot advance even though a main extension unit is extended;

FIG. 51C is an explanatory view illustrating a state of extending a second transforming portion in an inserting direction of an inserting portion by an operation of pushing out an elongated inserting portion of a medical instrument in the inserting direction of the inserting portion;

FIG. 52 is an explanatory view illustrating a primary part configuration of an insertion support tool according to a 23rd embodiment of the present invention;

FIG. 53A is a side view of a primary part showing a state where a roller fixing lever is engaged with a wire fixing portion of the insertion support tool according to the 23rd embodiment;

FIG. 53B is a plan view of FIG. 53A;

FIG. 53C is a side view of a primary part showing a state where the roller fixing lever has released engagement with the wire fixing portion;

FIG. 54A is a vertical cross-sectional view of a primary part showing a wire fixing state of a first modification of the wire fixing portion assembled in the insertion support tool according to the 23rd embodiment;

FIG. 54B is a cross-sectional view taken along a line 54B-54B in FIG. 54A;

FIG. 55A is a vertical cross-sectional view of a primary part showing a wire fixing released state where a wire fixing claw in the first modification of the wire fixing portion is broken off;

FIG. 55B is a front view of FIG. 55A;

FIG. 56A is a vertical cross-sectional view of a primary part showing a wire fixing state in a second embodiment of the wire fixing portion according to the 23rd embodiment;

FIG. 56B is a vertical cross-sectional view of a primary part showing a wire fixing released state;

FIG. 57 is a vertical cross-sectional view of a primary art showing a state where an end side extension unit of an insertion support tool according to a 24th embodiment of the present invention is not used;

FIG. 58 is a vertical cross-sectional view of a primary part showing a state where the end side extension unit of the insertion support tool according to the 24th embodiment is used;

FIG. 59 is an explanatory view illustrating a primary part configuration of an insertion support tool according to a 25th embodiment of the present invention;

FIG. 60 is a vertical cross-sectional view showing a primary part configuration of a driving device for the end side extension unit of the insertion support tool according to the 25th embodiment;

FIG. 61 is a side view showing a state where the end side extension unit of the insertion support tool according to the 25th embodiment is extended;

FIG. 62A is a schematic block diagram showing a state where the end side extension unit is extended at the time of an operation of the insertion support tool according to the 25th embodiment;

FIG. 62B is a schematic block diagram showing a state where the end side extension unit is contracted;

FIG. 63 is a vertical cross-sectional view showing a modification of a driving device of the end side extension unit of the insertion support tool according to the 25th embodiment;

FIG. 64 is vertical cross-sectional view of a primary part showing a state where an endoscope is assembled with respect to an insertion support tool according to a 26th embodiment of the present invention;

FIG. 65 is an explanatory view illustrating an operation of the insertion support tool according to the 26th embodiment;

FIG. 66 is a vertical cross-sectional view of a primary part showing a state where an endoscope is assembled with respect to an insertion support tool according to a 27th embodiment of the present invention;

FIG. 67 is an explanatory view illustrating an operation of the insertion support tool according to the 27th embodiment;

FIG. 68A is a vertical cross-sectional view of a primary part of an insertion support tool according to a 28th embodiment of the present invention;

FIG. 68B is an explanatory view illustrating an operation of a driving device for an end side extension unit according to the 28th embodiment;

FIG. 69 is a vertical cross-sectional view of a primary part showing an insertion support tool according to a 29th embodiment of the present invention; and

FIG. 70 is a side view showing the insertion support tool according to the 29th embodiment.

### DETAILED DESCRIPTION OF THE INVENTION

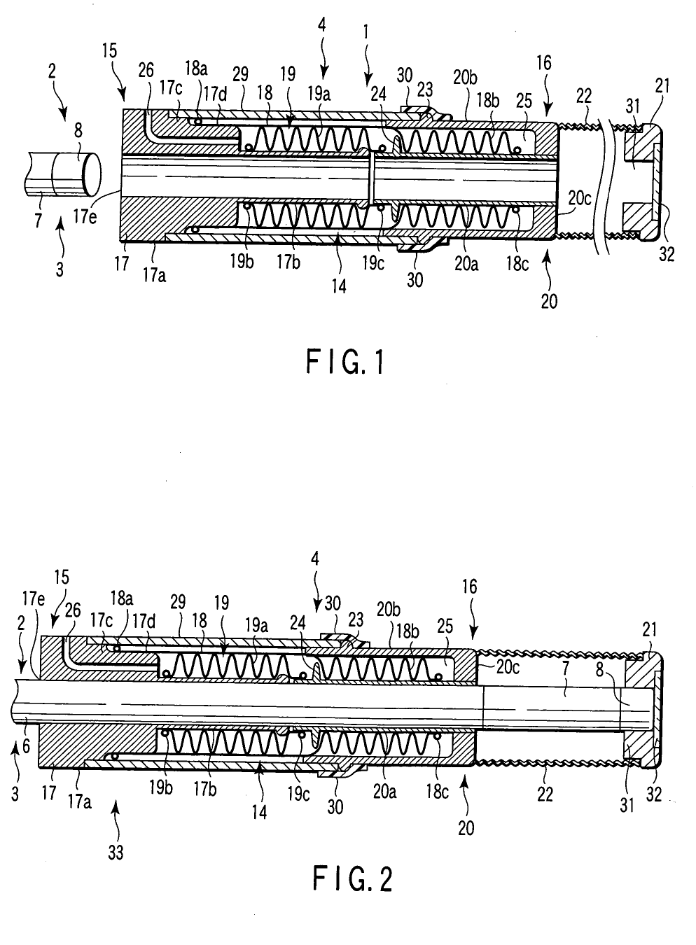

A first embodiment according to the present invention will now be described hereinafter with reference to FIGS. 1 to 7A. FIG. 1 shows an endoscope insertion apparatus 1 as an insertion apparatus for a medical instrument according to this embodiment. The endoscope insertion apparatus 1 has an insertion support tool 4 which is detachably attached at an end portion of an inserting portion 3 of a large-intestine endoscope 2.

As shown in FIG. 5, the large-intestine endoscope 2 has the elongated shaft-shaped inserting portion 3 which is inserted into a body and an operating portion with a large diameter which is arranged at a base end portion of the inserting portion 3. As shown in FIG. 2, the inserting portion 3 has an elongated flexible tube portion 6, a curving portion 7 which can curve in, e.g., four directions, i.e., up, down, right and left directions, and an end hard portion 8. Further, the base end portion of the flexible tube portion 6 is coupled with the operating portion 5. An end portion of a non-illustrated curving operation wire is coupled with the curving portion 7.

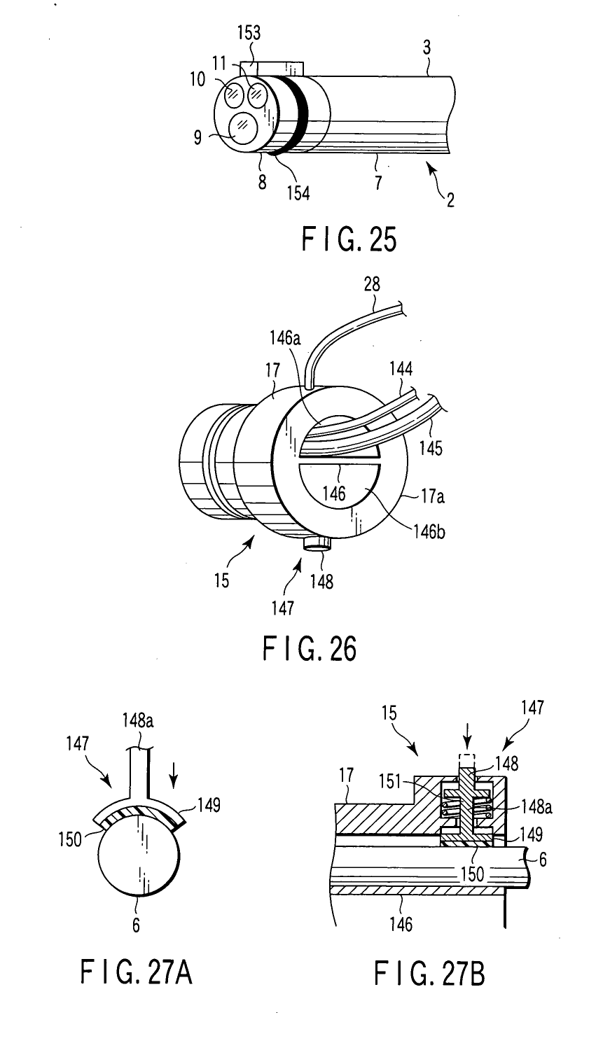

As shown in FIG. 25, an end surface of the end hard portion 8 has at least one observation window 9 and two illumination windows 10 and 11. An objective lens is arranged behind a cover glass of the observation window 9. An imaging element such as a CCD is arranged at an image forming position of this objective lens. One end portion of a signal cable is connected with this imaging element. An end portion of a light guide fiber bundle is arranged behind cover glasses of the illumination windows 10 and 11.

Furthermore, the signal cable of the imaging element, the light guide fiber bundle, the curving operation wire and others are extended to the operating portion 5 side through the inserting portion 3. Moreover, the signal cable of the imaging element, the light guide fiber bundle, the curving operation wire and others are packaged as built-in members in the inserting portion 3.

A curving operation knob 12 which operates the curving portion 7 is arranged on the operating portion 5. A central shaft of the curving operation knob 12 is coupled with a curving operation mechanism included in the operating portion 5. A base end portion of the curving operation wire is coupled with this curving operation mechanism. Additionally, the curving operation wire is towed by a rotating operation of the curving operation knob 12 via the curving operation mechanism, and the curving portion 7 is remotely operated in an operating direction of the curving operation knob 12.

Further, one end portion of a universal cord 13 is coupled with the operating portion 5. A light connector and an electric signal connector are provided at the other end portion of this universal cord 13. The light connector is connected with a light source device and the electric signal connector is connected with a video processor, respectively. Here, the signal cable or the light guide fiber bundle is inserted into the universal cord 13 from the operating portion 5. Furthermore, the signal cable is connected with the electric signal connector.

Moreover, the other end portion of the light guide fiber bundle is connected with the light connector. Illumination light emitted from a light source lamp of the light source device is led to the end hard portion 8 side of the inserting portion 3 through the light guide fiber bundle, and then emitted toward the outside from the illumination windows 10 and 11. Additionally, an observation image which has entered from the observation window 9 is converted into an electric signal by the imaging element at the image forming position of the objective lens. This electric signal is input to the video processor through the signal cable and signal-processed by the video processor, and a video signal is supplied to a non-illustrated monitor so that an image is displayed in a screen of the monitor.

Further, the insertion support tool 4 has a soft bag-shaped external sheath (a transforming portion) 14 which can be extended along an inserting direction of the inserting portion 3 of the large-intestine endoscope 2, a base unit 15 coupled with a base end portion of this external sheath 14, and an end unit 16 coupled with an end portion of the external sheath 14.

The base unit 15 has a substantially cylindrical mouthpiece member 17. The inserting portion 3 of the large-intestine endoscope 2 can be inserted into a cylinder of this mouthpiece member 17. A large-diameter portion 17a having the largest diameter is provided on an outer peripheral surface of the mouthpiece member 17 on the base end portion side thereof. An end cylindrical portion 17b having a diameter smaller than that of any other part is provided on an end portion side of the mouthpiece member 17. A diameter of a cylinder of this end cylindrical portion 17b is set to a dimension which allows insertion of the inserting portion 3 of the large-intestine endoscope 2.

Two step portions 17c and 17d having different outer diameters are formed between the large-diameter portion 17a and the end cylindrical portion 17b of the mouthpiece member 17. The large-diameter step portion 17c having a large diameter is arranged on the base end portion side of the mouthpiece member 17, and the small-diameter step portion 17d having a diameter smaller than that of the large-diameter step portion 17c is arranged on the end portion side apart from this large-diameter step portion 17c.

Furthermore, the external sheath 14 has an outer sheath 18 and an inner sheath 19. A rear side fixing ring 18a is formed at a base end portion of the outer sheath 18. The rear side fixing ring 18a is fixed at the small-diameter step portion 17d of the mouthpiece member 17. Moreover, an end side of the outer sheath 18 is extended toward the end unit 16 side. Additionally, a folded first bellows portion 18b is formed on this extended portion side.

A folded second bellows portion 19a is formed in the inner sheath 19. This second bellows portion 19a is arranged on the end cylindrical portion 17b of the mouthpiece member 17. Further, a rear side fixing ring 19b which is fixed at a base end portion of the end cylindrical portion 17b is formed at a base end portion of this second bellows portion 19a.

The end unit 16 has an external sheath fixing member 20, a hard end member 21 and a soft coupling tube 22. The external sheath fixing member 20 has an inner cylindrical portion 20a whose diameter is substantially the same as that of the end cylindrical portion 17b of the mouthpiece member 17, and an outer cylindrical portion 20b whose diameter is larger than that of this inner cylindrical portion 20a. An outer diameter of the outer cylindrical portion 20b is set to be substantially equal to that of the large-diameter step portion 17c of the mouthpiece member 17.

A bent portion 20c which is inwardly bent is formed at an end portion of the outer cylindrical portion 20b. An inner end portion of this bent portion 20c is fixed on an outer peripheral surface of the inner cylindrical portion 20a. Furthermore, an annular first flange portion 23 having a large diameter is formed to protrude on an outer peripheral surface of the outer cylindrical portion 20b on a base end portion side thereof.

An annular second flange portion 24 having a large diameter is formed to protrude on an outer peripheral surface of the inner cylindrical portion 20a on a base end portion side thereof. Moreover, an outer sheath accommodation space 25 is formed in front of the second flange portion 24 between the inner cylindrical portion 20a and the outer cylindrical portion 20b. The first bellows portion 18b of the outer sheath 18 is accommodated in this outer sheath accommodation space 25. A front side fixing ring 18c which is fixed at the end portion of the inner cylindrical portion 20a is formed at an end portion of this first bellows portion 18b. A front side fixing ring 19c which is fixed on the outer peripheral surface of the inner cylindrical portion 20a is formed at the end portion of the second bellows portion 19a in the inner sheath 19. This front side fixing ring 19c is arranged on a rear side apart from the second flange portion 24 of the inner cylindrical portion 20a.

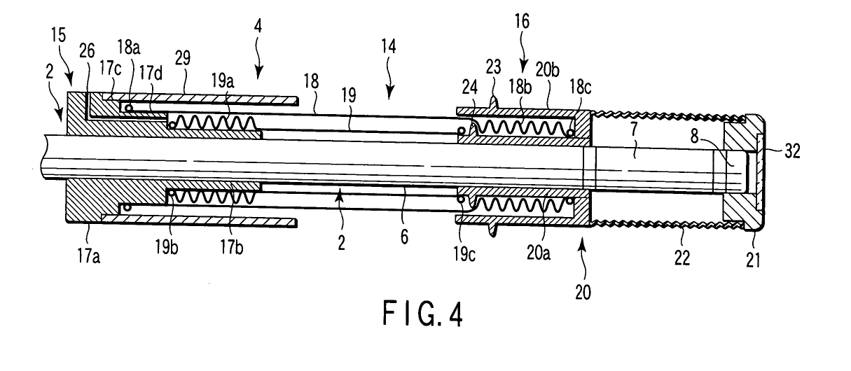

Additionally, a fluid path 26 through which a working fluid such as pressurized air is supplied is formed in the mouthpiece member 17 between the outer sheath 18 and the inner sheath 19 of the external sheath 14. An inner end portion of this fluid path 26 communicates with a part between the outer sheath 18 and the inner sheath 19 of the mouthpiece member 17. Further, an outer end portion of the fluid path 26 is coupled with an air supply mouthpiece 27 arranged on the outer peripheral surface of the mouthpiece member 17 as shown in FIG. 5. One end portion of an air supply tube 28 is coupled with this air supply mouthpiece 27. The other end portion of this air supply tube 28 is coupled with a non-illustrated air supply pump. Furthermore, a working fluid such as pressurized air is supplied to a part between the outer sheath 18 and the inner sheath 19 from this air supply tube 28 through the air supply mouthpiece 27 and the fluid path 26 when the external sheath 14 of the insertion support tool 4 is extended. At this time, the external sheath 14 is extended in the inserting direction of the inserting portion 3 by a supply pressure of the working fluid (see FIG. 4).

Moreover, a cylindrical hard cover member 29 is arranged around the external sheath 14. This cover member 29 is formed of a hard material such as a hard rubber material, a plastic material, a composite material in which a soft rubber material covers a surface of a cored bar, or the like. A base end portion of the cover member 29 is fixed at the large-diameter step portion 17c of the mouthpiece member 17. An end portion of this cover member 29 is extended toward the end unit 16 side. Additionally, as shown in FIG. 1, when the external sheath 14 is contracted, the end portion of the cover member 29 is maintained in a state where it is in contact with the first flange portion 23 of the end unit 16. In this state, the cover member 29 keeps covering the external sheath 14. A length of the cover member 29 is set to a length which allows the end portion of the cover member 29 to enter a large intestine (a rectum) H2 beyond an anus H1, e.g., 3 cm or below when the insertion support tool 4 is inserted into the large-intestine (the rectum) H2 from the anus H1 of a patient H.

Further, to the insertion support tool 4 is provided an adhesive tape (engagement means) 30 as a holding member which holds the end portion of the cover member 29 in a state where this end portion is in contact with the first flange portion 23 of the end unit 16. An end portion side of this adhesive tape 30 is attached and fixed at the end unit 16, and a base end portion side of the same is attached and fixed at the cover member 29. As a result, when the external sheath 14 is contracted, this adhesive tape 30 maintains an engaged state where the end portion of the cover member 29 is releasably engaged with the end unit 16.

Furthermore, the end member 21 has an endoscope attachment hole 31 at a substantial shaft center portion thereof. A front side of this endoscope attachment hole 31 is sealed by a transparent cover glass 32. Moreover, the end hard portion 8 of the inserting portion 3 of the large-intestine endoscope 2 can be coupled with the endoscope attachment hole 31 of the end member 21 in a fitting state.

The soft coupling tube 22 is stretched between the end member 21 and the outer cylindrical portion 20b of the external sheath fixing member 20. This coupling tube 22 is arranged at a position corresponding to the curving portion 7 of the large-intestine endoscope 2 in a state where the insertion support tool 4 is assembled with respect to the end portion of the inserting portion 3 of the large-intestine endoscope 2. Therefore, the coupling tube 22 can freely transform in accordance with a curving operation of the curving portion 7 of the large-intestine endoscope 2.

A function of the above-mentioned structure will now be described. The endoscope insertion apparatus 1 according to this embodiment is used in a state where the insertion support tool 4 is attached to the inserting portion 3 of the large-intestine endoscope 2 when utilizing the large-intestine endoscope 2.

FIG. 1 shows a state before the insertion support tool 4 is assembled with respect to the end portion of the inserting portion 3 of the large-intestine endoscope 2. At this time, the insertion support tool 4 is maintained in a state where the external sheath 14 is previously contracted and the hard cover member 29 is arranged on the outer side of the external sheath 14 to cover the external sheath 14. Here, the external sheath 14 is maintained in a state where the second bellows portion 19a of the inner sheath 19 is folded and arranged on the end cylindrical portion 17b of the mouthpiece member 17 in the folded manner and the first bellows portion 18b of the outer sheath 18 is folded and accommodated in the outer sheath accommodation space 25 between the inner cylindrical portion 20a and the outer cylindrical portion 20b of the external sheath fixing member 20 in the folded manner. Additionally, the adhesive tape 30 maintains an engaged state where the end portion of the cover member 29 is releasably engaged with the end unit 16. In this state, there is carried out an operation of attaching the insertion support tool 4 to the inserting portion 3 of the large-intestine endoscope 2 (an insertion support tool attachment step).

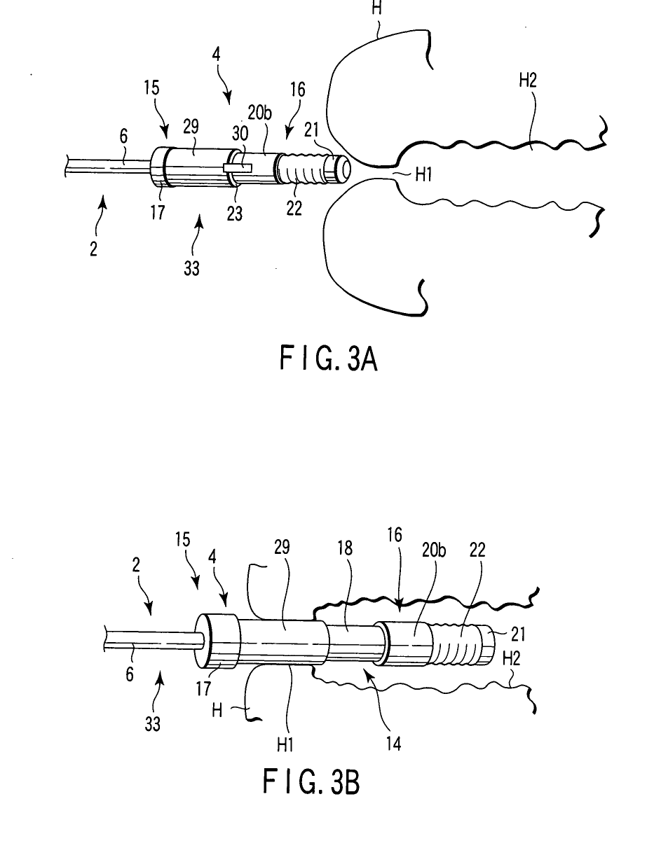

In this insertion support tool attachment operation, the inserting portion 3 of the large-intestine endoscope 2 is inserted from the end side thereof into the cylinder of the mouthpiece member 17 from an opening portion 17e on a base end surface of the mouthpiece member 17 of the insertion support tool 4. Further, the end hard portion 8 of the inserting portion 3 is coupled with the endoscope attachment hole 31 of the end member 21 in a fitting state as shown in FIG. 2, and an assembled unit 33 having the insertion support tool 4 assembled at the end part of the inserting portion 3 of the large-intestine endoscope 2 is set as shown in FIG. 3A.

After attachment of this insertion support tool 4, the assembled unit 33 is inserted into the large intestine (the rectum) H2 from the anus H1 of the patient H (a first insertion step). In this inserting operation, the insertion support tool 4 is inserted into the anus H1 of the patient H from the end member 21 side of the insertion support tool 4. At this time, since the anus H1 of the patient H is usually closed by a sphincter muscle, the assembled unit 33 is pushed into the anus H1 of the patient H while spreading this sphincter muscle of the anus H1.

Furthermore, during the operation of inserting the assembled unit 33, the adhesive tape 30 is unstuck from the insertion support tool 4 when the end portion of the adhesive tape 30 has come close to the anus H1. As a result, engagement between the end portion of the cover member 29 and the end unit 16 is released.

Thereafter, the operation of pushing the assembled unit 33 into the anus H1 of the patient H is continued. At this time, since the end portion of the cover member 29 is in contact with the first flange portion 23 of the end unit 16, a pushing force from the cover member 29 side is transmitted to the end unit 16 through the first flange portion 23. Therefore, the operation of inserting the assembled unit 33 in which the insertion support tool 4 is assembled together with the inserting portion 3 of the large-intestine endoscope 2 is continued like before the adhesive tape 30 is unstuck.

Moreover, as shown in FIG. 3B, the assembled unit 33 is inserted to a position at which the end portion of the cover member 29 of the insertion support tool 4 passes the anus H1. In this state, the cover member 29 of the insertion support tool 4 is fixed in the anus H1 by a fastening force of the sphincter muscle of the anus H1.

Additionally, after the cover member 29 of the insertion support tool 4 is fixed in the anus H1, a working fluid such as pressurized air is supplied from the air supply tube 28 to a part between the outer sheath 18 and the inner sheath 19 through the air supply mouthpiece 27 and the fluid path 26. At this time, a space between the outer sheath 18 and the inner sheath 19 of the external sheath 14 is inflated by supply of the working fluid. Therefore, as shown in FIG. 4, the entire external sheath 14 is extended in the inserting direction of the inserting portion 3 of the large-intestine endoscope 2. As a result, the insertion support tool 4 moves forward in a state where the end unit 16 is pushed frontward with respect to the base unit 15. At this time, the inserting portion 3 of the large-intestine endoscope 2 advances together with the end unit 16 in the inserting direction of the inserting portion 3 (a second insertion step).

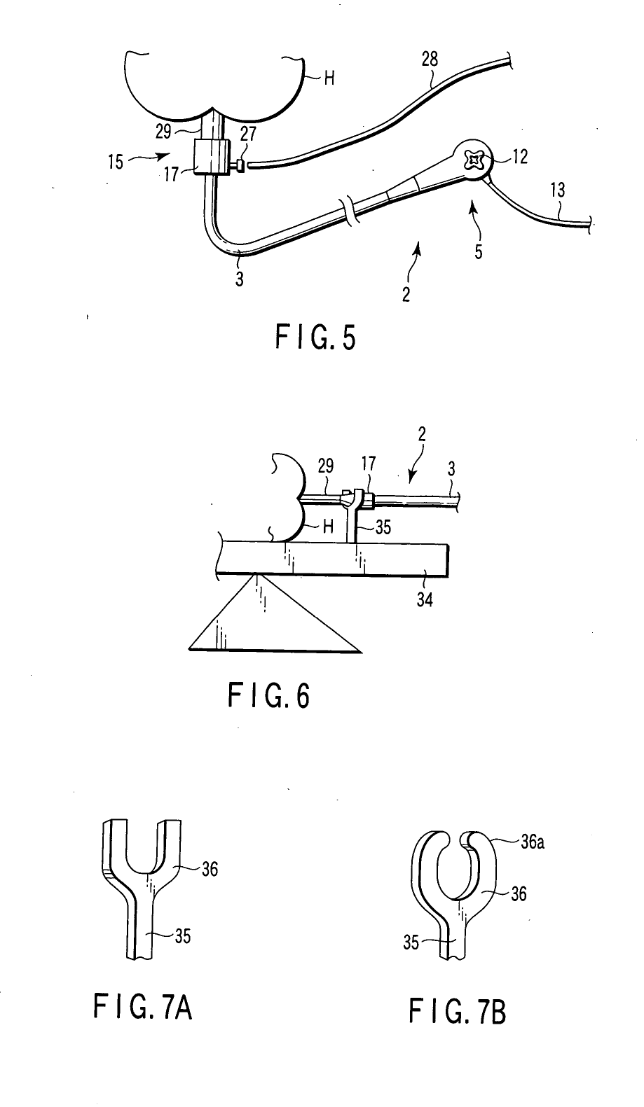

Further, FIG. 6 shows a state in which the assembled unit 33 having the insertion support tool 4 assembled with respect to the inserting portion 3 of the large-intestine endoscope 4 is inserted into the patient H lying down on a bed 34. Here, a stand 35 for supporting a medical instrument is provided on the bed 34. A substantially U-shaped support frame 36 is provided at an upper end portion of this stand 35 as shown in FIG. 7A. Furthermore, the mouthpiece member 17 of the insertion support tool 4 is supported in a state where it is releasably engaged with the support frame 36 of the stand 35. It is to be noted that, like a modification shown in FIG. 7B, a folded portion 36a for retention having elasticity may be provided to the support frame 36 of the stand 35 on an opening end portion side.

Thus, the apparatus having the above-described configuration demonstrates the following effects. That is, in the endoscope insertion apparatus 1 according to this embodiment, attaching the insertion support tool 4 to the inserting portion 3 of the large-intestine endoscope 2 can extend the soft external sheath 14 of the insertion support tool 4 in the inserting direction of the inserting portion 3 when inserting the inserting portion 3 of the large-intestine endoscope 2, thus supporting the inserting operation of the inserting portion 3 of the large-intestine endoscope 2. Therefore, the operation of inserting the inserting portion 3 of the large-intestine endoscope 2 into the large intestine (the rectum) H2 of the patient H can be facilitated.

Moreover, when the external sheath 14 of the insertion support tool 4 is contracted, the hard cover member 29 on the outer side of the external sheath 14 can be maintained to cover the external sheath 14. Therefore, at the time of the operation of attaching the insertion support tool 4 to the inserting portion 3 of the large-intestine endoscope 2, this operation can be performed in a state where the hard cover member 29 is grasped. As a result, since a force of an operator who holds the insertion support tool 4 by hand does not directly act on the external sheath 4 during the operation of attaching the insertion support tool 4, the external sheath 14 is not unnecessarily extended. As a result, the operation of attaching the insertion support tool 4 to the inserting portion 3 of the large-intestine endoscope 2 can be facilitated.

Additionally, at the time of the operation of inserting the assembled unit 33 in which the insertion support tool 4 is assembled with respect to the end part of the inserting portion 3 of the large-intestine endoscope 2 into the large intestine (the rectum) H2 from the anus H1 of the patient H, the fastening force of the sphincter muscle of the anus H1 can be received by the cover member 29 of the insertion support tool 4. Therefore, during the operation of inserting the assembled unit 33 into the anus H1 of the patient H, the soft external sheath 14 is not crushed or bent by the fastening force of the sphincter muscle of the anus H1. As a result, the operation of inserting the assembled unit 33 into the anus H1 of the patient H can be smoothly and assuredly performed. Further, since the external sheath 14 is not exposed in a state where the external sheath 14 of the insertion support tool 4 is contracted, the external sheath 14 is not damaged before use.

Furthermore, in this embodiment, to the insertion support tool 4 is provided the adhesive tape 30 which holds the end portion of the cover member 29 in a state where this end portion is in contact with the first flange portion 23 of the end unit 16. Therefore, since the external sheath 14 is not unnecessarily extended during the operation of attaching the insertion support tool 4 to the inserting portion 3 of the large-intestine endoscope 2, the operation of attaching the insertion support tool 4 to the inserting portion 3 of the large-intestine endoscope 2 can be further readily carried out.

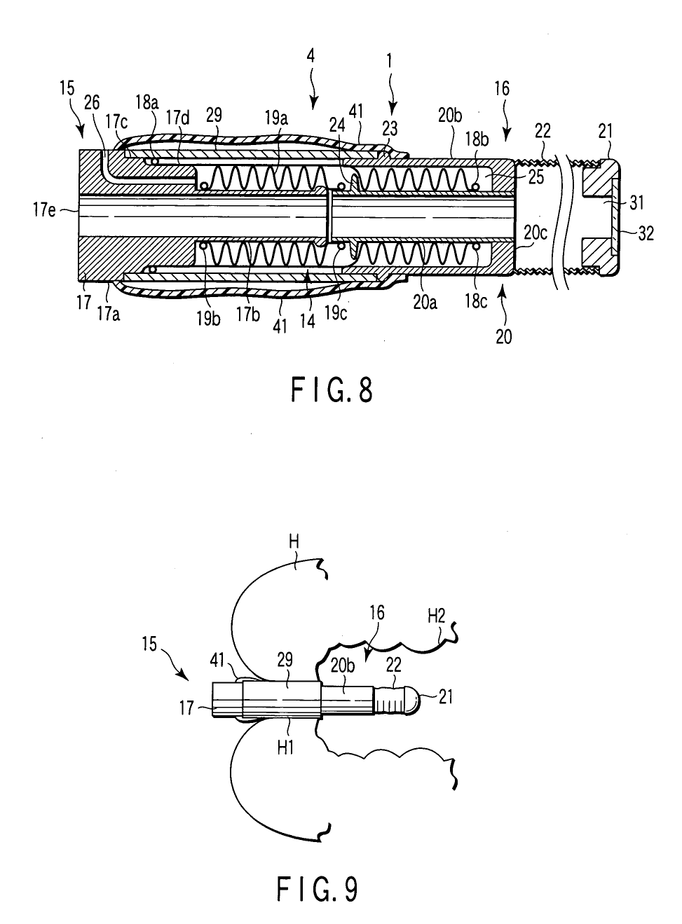

FIGS. 8 and 9 show a second embodiment according to the present invention. In this embodiment, the configuration of the insertion support tool 4 of the endoscope insertion apparatus 1 according to the first embodiment (see FIGS. 1 to 7A) is changed as follows.

That is, the first embodiment shows a configuration using the adhesive tape 30 as engagement means which holds the end portion of the cover member 29 in a state where this end portion is in contact with the first flange portion 23 of the end unit 16. On the contrary, an insertion support tool 4 according to this embodiment has a configuration in which a fixed thread 41 is used in place of this adhesive tape 30. An end portion of this fixed thread 41 is fixed at an outer cylindrical portion 20b of an external sheath fixing member 20 of an end unit 16. A base end portion of this fixed thread 41 is fixed at a mouthpiece member 17 or a rear end portion of a cover member 29. When attaching this fixed thread 41, the fixed thread 41 is pulled and fixed in a state where an end portion of the cover member 29 is in contact with a first flange portion 23 of the end unit 16. As a result, even in a state where the fixed thread 41 is fully stretched, the cover member 29 and the end unit 16 are not separated from each other.

A function of this embodiment having the above-mentioned configuration will now be described. In this embodiment, in the insertion support tool 4, an external sheath 14 is previously contracted before use. Additionally, the hard cover member 29 is maintained arranged on the outer side of the external sheath 14 in a state where the external sheath 14 is covered. At this time, the fixed thread 41 maintains an engagement state in which the end portion of the cover member 29 is releasably engaged with the end unit 16. In this state, there are carried out the operation of attaching the insertion support tool 4 to the inserting portion 3 of the large-intestine endoscope 2 (an insertion support tool attachment step) and the operation of inserting the assembled unit 33 in which the insertion support tool 4 is assembled with respect to the end portion of the inserting portion 3 of the large-intestine endoscope 2 into the large intestine (the rectum) H2 from the anus H1 of the patient H (a first insertion step).

Further, during the operation of inserting the assembled unit 33, as shown in FIG. 9, the end portion of the cover member 29 is inserted into the anus H1, and then the fixed thread 41 is cut when the base end portion of the fixed thread 41 has come close to the anus H1. As a result, engagement between the end portion of the cover member 29 and the end unit 16 is released.

Thereafter, supply of a working fluid such as pressurized air to a part between the outer sheath 18 and the inner sheath 19 of the external sheath 14 can extend the entire external sheath 14 in the inserting direction of the inserting portion 3 of the large-intestine endoscope 2. As a result, the insertion support tool 4 moves forward in a state where the end unit 16 is pushed forward with respect to the base unit 15. At this time, the inserting portion 3 of the large-intestine endoscope 2 is moved forward together with the end unit 16 in the inserting direction of the inserting portion 3 (a second insertion step).

Thus, the apparatus having the above-described configuration demonstrates the following effects. That is, in the endoscope insertion apparatus 1 according to this embodiment, the fixed thread 41 is used as engagement means for holding the end portion of the cover member 29 in a state where this end portion is in contact with the first flange portion 23 of the end unit 16. Therefore, during the operation of attaching the insertion support tool 4 to the inserting portion 3 of the large-intestine endoscope 2, the external sheath 14 is not unnecessarily extended, and hence it is possible to readily perform the operation of attaching the insertion support tool 4 to the inserting portion 3 of the large-intestine endoscope 2 and the operation of inserting the assembled unit 33 having the insertion support tool 4 assembled with respect to the end portion of the inserting portion 3 of the large-intestine endoscope 2 into the large intestine (the rectum) H2 from the anus H1 of the patient H (the first insertion step) like the first embodiment.

Furthermore, in a state where the external sheath 14 of the insertion support tool 4 is contracted, the soft external sheath 14 is covered with the cover member 29. Therefore, since the soft external sheath 14 is not exposed to the outside, the external sheath 14 is not damaged before use.

Moreover, FIG. 10 shows a third embodiment according to the present invention. In this embodiment, the configuration of the insertion support tool 4 of the endoscope insertion apparatus 1 according to the first embodiment (see FIGS. 1 to 7A) is changed as follows.

That is, in this embodiment, an annular engagement protrusion 51 which protrudes in an inner direction is provided on an end portion inner peripheral surface of a cover member 29 of an insertion support tool 4. Additionally, an annular locking concave portion 52 is formed on a base end portion outer peripheral surface of an outer cylindrical portion 20b of an end unit 16 behind a first flange portion 23. Further, a rear end flange portion 53 having a diameter larger than that of the locking concave portion 52 is protrudingly provided behind this locking concave portion 52.

Furthermore, the engagement protrusion 51 of the cover member 29 of the insertion support tool 4 is fitted in the locking concave portion 52 of the outer cylindrical portion 20b of the end unit 16, thereby forming a concavoconvex fitting portion 54. This concavoconvex fitting portion 54 forms engagement means for holding the end portion of the cover member 29 in a state where this end portion is in contact with the first flange portion 23 of the end unit 16.

A function of this embodiment having the above-mentioned structure will now be described. In this embodiment, the insertion support tool 4 is maintained in a state where an external sheath 14 is previously contracted before use and the hard cover member 29 is arranged on the outer side of the external sheath 14 to cover the external sheath 14. At this time, the engagement protrusion 51 of the cover member 29 of the insertion support tool 4 moves over the rear end flange portion 53 of the outer cylindrical portion 20b of the end unit 16 to be fitted in the locking concave portion 52, thereby forming the concavoconvex fitting portion 54. This concavoconvex fitting portion 54 maintains an engagement state in which the end portion of the cover member 29 is releasably engaged with the end unit 16. In this state, there are carried out an operation of attaching the insertion support tool 4 to the inserting portion 3 of the large-intestine endoscope 2 (an insertion support tool attachment step) and an operation of inserting an assembled unit 33 having the insertion support tool 4 assembled at the end portion of the inserting portion 3 of the large-intestine endoscope 2 into a large intestine (a rectum) H2 from the anus H1 of a patient H (a first insertion step).

Furthermore, in regard to engagement strength of the concavoconvex fitting portion 54 between the engagement protrusion 51 of the cover member 29 and the locking concave portion 52 of the outer cylindrical portion 20b of the end unit 16, it is preferable to set a working fluid such as pressurized air from a fluid path 26 to a pressure of air or below supplied to a part between an outer sheath 18 and an inner sheath 19 of the external sheath 14. In this case, start of an extending operation of the external sheath 14 by supply of the working fluid such as pressurized air from the fluid path 26 releases engagement between the end portion of the cover member 29 and the end unit 16.

Thereafter, supplying the working fluid such as pressurized air to the space between the outer sheath 18 and the inner sheath 19 of the external sheath 14 can extend the entire external sheath 14 in an inserting direction of the inserting portion 3 of the large-intestine endoscope 2. As a result, the insertion support tool 4 can move forward in a state where the end unit 16 is pushed forward with respect to a base unit 15. At this time, the inserting portion 3 of the large-intestine endoscope 2 is moved forward together with the end unit 16 in the inserting direction of the inserting portion 3 (a second insertion step).

Thus, the apparatus having the above-described configuration demonstrates the following effects. That is, in the endoscope insertion apparatus 1 according to this embodiment, the engagement protrusion 51 of the cover member 29 of the insertion support tool 4 moves over the rear end flange portion 53 of the outer cylindrical portion 20b of the end unit 16 to be fitted in the locking concave portion 52, whereby the end portion of the cover member 29 can be engaged with the end unit 16. Therefore, it is possible to readily carry out the engagement operation of holding the end portion of the cover member 29 in a state where this end portion is in contact with the first flange portion 23 of the end unit 16.

FIG. 11 shows a fourth embodiment according to the present invention. This embodiment can be obtained by changing the configuration of the insertion support tool 4 of the endoscope insertion apparatus 1 according to the first embodiment (see FIGS. 1 to 7A) as follows.

That is, in this embodiment, a barb portion 61 having a diameter larger than that of any part other than an end portion of a cover member 29 is provided at the end portion of the cover member 29 of an insertion support tool 4. This barb portion 61 is obtained by smoothing the end portion of the cover member 29 and increasing a diameter of the same. A bent portion 62 which is bent in a direction substantially orthogonal to an axial direction of the insertion support tool 4 is formed at a base end portion of this barb portion 61.

A function of the above-mentioned configuration will now be described. In this embodiment, at the time of an operation of inserting an assembled unit 33 having the insertion support tool 4 assembled at the end portion of the inserting portion 3 of the large-intestine endoscope 2 into the large intestine (rectum) H2 from the anus H1 of a patient H (a first insertion step), the assembled unit 33 is pushed in until the barb portion 61 of the cover member 29 moves over the anus H1 of the patient H. At this time, when the barb portion 61 of the cover member 29 moves past the anus H1 of the patient H, the force needed is suddenly reduced. Therefore, at the time of the operation of inserting the assembled unit 33 into the large intestine (the rectum) H2 from the anus H1 of the patient H, it is possible to relatively easily confirm, from an operational feeling of an operator, when the barb portion 61 of the cover member 29 moves past the anus H1 of the patient H by a change in the force needed for pushing in the assembled unit 33.

Thus, the apparatus having the above-described configuration demonstrates the following effect. That is, in the endoscope insertion apparatus 1 according to this embodiment, it is possible to confirm from an operational feeling of an operator when the barb portion 61 of the cover member 29 moves past the anus H1 of the patient H during the operation of inserting the assembled unit 33 into the large intestine (the rectum) H2 from the anus H1 of the patient H. Therefore, since the insertion support tool 4 can be assuredly inserted to a correct insertion position, it is possible to further readily perform the operation of inserting the assembled unit 33 having the insertion support tool 4 assembled at the end portion of the inserting portion 3 of the large-intestine endoscope 2 into the large intestine (the rectum) H2 from the anus H1 of the patient H (the first insertion step).

Moreover, when the insertion support tool 4 has been inserted into the large intestine (the rectum) H2 from the anus H1 of the patient H, as shown in FIG. 11, a step portion between the bent portion 62 of the barb portion 61 and the outer peripheral surface of the cover member 29 can be brought into contact with a wall surface between the inside of the large intestine (the rectum) H2 and the anus H1. Therefore, the insertion support tool 4 which has been inserted into the large intestine (the rectum) H2 can be prevented from being easily pulled out from the large intestine (the rectum) H2 during the operation of inserting the inserting portion 3 of the large-intestine endoscope 2.

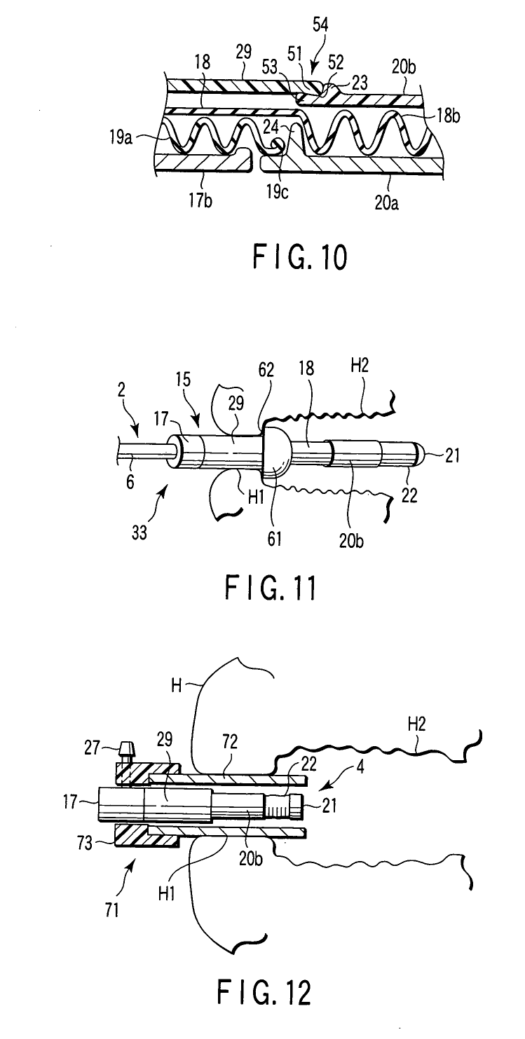

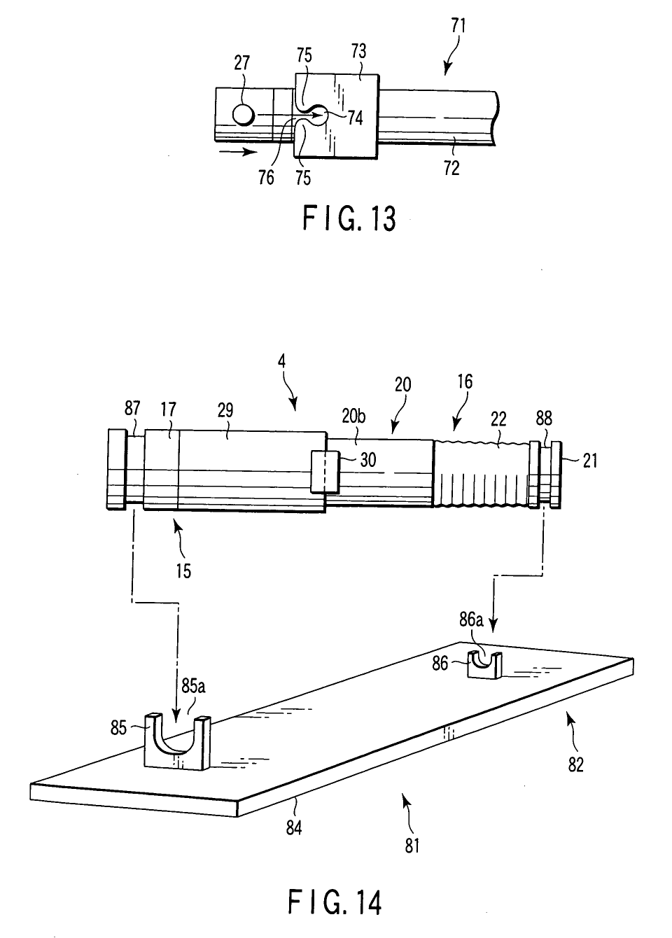

FIGS. 12 and 13 show a fifth embodiment according to the present invention. This embodiment is obtained by changing the configuration of the endoscope insertion apparatus 1 of the first embodiment (see FIGS. 1 to 7A) as follows.

That is, in this embodiment, there is provided a protection member 71 which is previously inserted into the large intestine (rectum) H2 from the anus H1 of a patient H and attached in a muzzle-loaded state before inserting a main body of an insertion support tool 4 as shown in FIG. 12. This protection member 71 has a cylindrical member 72 formed of a hard material and a large-diameter mouthpiece member 73 formed of a resin which is coupled with a base end portion of this cylindrical member 72.

As shown in FIG. 13, in the mouthpiece member 73, a mouthpiece engagement groove 74 as a fixing portion at which at least a part of the insertion support tool 4 is fixed is formed on a circumferential wall surface of the mouthpiece member 73 on a base end portion side thereof. This mouthpiece engagement groove 74 is formed into a shape which can be engaged with an air supply mouthpiece 27 of a mouthpiece member 17 of the insertion support tool 4.

An engagement protrusion 75 which protrudes toward an inner side of the mouthpiece engagement groove 74 is formed on each of both sides of an opening end portion (a left end portion in FIG. 13) of the mouthpiece engagement groove 74. A narrow-width portion 76 whose width is narrower than a groove width of the mouthpiece engagement groove 74 is formed between the engagement protrusions 75 on both sides.

Further, when an endoscope insertion apparatus 1 according to this embodiment is used, an assembled unit 33 having the insertion support tool 4 assembled at an end portion of an inserting portion 3 of a large-intestine endoscope 2 according to the first embodiment is inserted into the protection member 71.

A function of this embodiment having the above-mentioned configuration will now be described. In this embodiment, when the large-intestine endoscope 2 is used, the cylindrical member 72 of the protection member 71 is previously inserted into the large intestine (the rectum) H2 from the anus H1 of the patient H and attached in a muzzle-loaded state. Then, the assembled unit 33 having the insertion support tool 4 assembled at the end portion of the inserting portion 3 of the large-intestine endoscope 2 according to the first embodiment is inserted into the protection member 71. When inserting this insertion support tool 4, as indicated by an arrow in FIG. 13, the air supply mouthpiece 27 of the mouthpiece member 17 covers the narrow-width portion 76 from the opening end portion of the mouthpiece engagement groove 74 to be engaged with the mouthpiece engagement groove 74. As a result, the insertion support tool 4 can be prevented from rotating in a periaxial direction or from coming off with respect to the protection member 71.

Thus, the apparatus having the above-described configuration demonstrates the following effects. That is, in the endoscope insertion apparatus 1 according to this embodiment, the protection member 71 is provided separately from the insertion support tool 4, and the cylindrical member 72 of the protection member 71 is previously inserted into the large intestine (the rectum) H2 from the anus H1 of the patient H and attached in a muzzle-loaded state before inserting the main body of the insertion support tool 4. Therefore, the main body of the insertion support tool 4 can be protected by this protection member 71 in the muzzle-loaded state, thereby reducing a length of the cover member 29 of the insertion support tool 4. As a result, the insertion support tool 4 can be relatively reduced in dimension and configured to be compact, thus improving handling properties or attachment properties with respect to the large-intestine endoscope 2.

Furthermore, since the main body of the insertion support tool 4 can be inserted into the large intestine (the rectum) H2 from the anus H1 of the patient H by just performing the simple operation of inserting the main body of the insertion support tool 4 into the cylindrical member 72 of the protection member 71, it is possible to readily effect the operation of inserting the insertion support tool 4 into the large intestine (the rectum) H2 from the anus H1 of the patient H.

FIGS. 14 to 16 show a sixth embodiment according to the present invention. This embodiment is obtained by changing the configuration of the insertion support tool 4 of the endoscope insertion apparatus 1 according to the first embodiment (see FIGS. 1 to 7A) as follows.

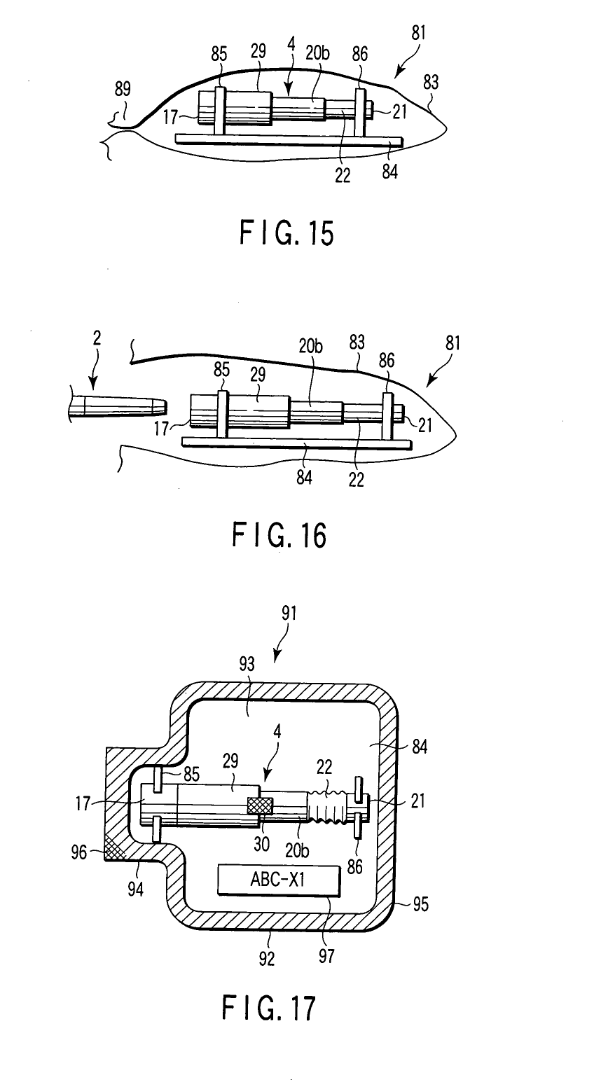

That is, in this embodiment, a packing material 81 which is used to pack an insertion support tool 4 is provided as shown in FIG. 15. This packing material 81 has a pedestal unit 82 shown in FIG. 14 and a packing bag 83 depicted in FIG. 15. The pedestal unit 82 has a tabular pedestal 84, and a pair of (first and second) support leg portions 85 and 86 standing on this pedestal 84. A substantially U-shaped first engagement groove 85a is formed in the first support leg portion 85. Likewise, a substantially U-shaped engagement groove 86a is formed in the second support leg portion 86.

Moreover, in the insertion support tool 4 according to this embodiment, a first annular groove 87 is formed on an outer peripheral surface of a mouthpiece member 17 of a base unit 15 and a second annular groove 88 is formed on an outer peripheral surface of an end member 21 of an end unit 16, respectively. The first annular groove 87 of the mouthpiece member 17 is releasably engaged in the first engagement groove 85a of the first support leg portion 85 on the pedestal 84 and the second annular groove 88 of the end member 21 is releasably engaged in the second engagement groove 86a of the second support leg portion 86 on the same, respectively. Additionally, the insertion support tool 4 on the pedestal 84 is restricted to a fixed set length between front and rear parts thereof and a fixed shape by engagement of the first and second support leg portions 85 and 86. As a result, an external sheath 14 is contracted and held in a state where a cover member 29 covers the external sheath 14.

A packing bag 83 is formed of a sterile bag in which an entire assembled unit including the pedestal unit 82 and the insertion support tool 4 is accommodated in a state where the insertion support tool 4 is held on the pedestal 84. An unsealing portion 89 is formed at one end portion of this packing bag 83. Further, the insertion support tool 4 is accommodated in a state where the mouthpiece member 17 of the base unit 15 is arranged on the unsealing portion 89 side.

A function of this embodiment having the above-mentioned configuration will now be described. In the endoscope insertion apparatus 1 according to this embodiment, there is carried out an operation of assembling an end portion of an inserting portion 3 of a large-intestine endoscope 2 with respect to the insertion support tool 4 in a state where the entire assembled unit including the pedestal unit 82 and the insertion support tool 4 is accommodated in the packing bag 83. At the time of this operation, as shown in FIG. 16, the unsealing portion 89 of the packing bag 83 is opened. At this time, the mouthpiece member 17 of the insertion support tool 4 is exposed from the unsealing portion 89 of the opened packing bag 83. Therefore, the end portion of the inserting portion 3 of the large-intestine endoscope 2 can be inserted from the mouthpiece member 17 of the insertion support tool 4 in a state where the insertion support tool 4 is within the packing bag 83. At this time, since the insertion support tool 4 is fixed on the pedestal 84, the insertion support tool 4 is not moved. Therefore, an operator can insert the end portion of the inserting portion 3 of the large-intestine endoscope 2 from the mouthpiece member 17 of the insertion support tool 4 through the packing bag 83 and then assemble the end portion of the inserting portion 3 of the large-intestine endoscope 2 with respect to the insertion support tool 4 without directly touching the insertion support tool 4.

Thus, the apparatus having the above-described configuration demonstrates the following effects. That is, in this embodiment, the packing material 81 which is used to pack the insertion support tool 4 is provided, and the packing bag 83 is formed of a sterile bag which accommodates the entire assembled unit including the pedestal unit 82 and the insertion support tool 4 in a state where the insertion support tool 4 is held on the pedestal 84. The insertion support tool 4 on the pedestal 84 is restricted to a fixed set length between the front and rear sides and a shape by engagement between the first and second support leg portions 85 and 86. As a result, the insertion support tool 4 is held in a state where the external sheath 14 is contracted and the cover member 29 covers the external sheath 14, and hence the external sheath 14 of the insertion support tool 4 can be prevented from being extended during transport. Therefore, an inconvenience of the insertion support tool 4 before use can be avoided.

Further, an operator can perform an operation of assembling the end portion of the inserting portion 3 of the large-intestine endoscope 2 with respect to the insertion support tool 4 through the packing bag 83 without directly touching the insertion support tool 4 with his/her hand. As a result, the external sheath 14 can be prevented from being extended or attached at the time of taking out or attaching the insertion support tool 4, thereby improving the handling properties of the insertion support tool 4. Therefore, it is possible to improve the workability of the assembling operation of assembling the end portion of the inserting portion 3 of the large-intestine endoscope 2 with respect to the insertion support tool 4.

FIG. 17 shows a first modification of the packing bag 83 of the insertion support tool 4 of the endoscope insertion apparatus 1 according to the sixth embodiment (see FIGS. 14 to 16). A packing bag 91 according to this modification has a box-shaped packing bag main body 92 whose upper surface is opened and a sealing film 93 which covers the upper surface opening portion of this packing bag main body 92.

Moreover, a narrow-width portion 94 is formed at a part of the packing bag main body 92. This narrow-width portion 94 is provided at a position corresponding to a mouthpiece member 17 of an insertion support tool 4. Additionally, a sealing portion 95 of the sealing film 93 is formed on an entire circumference of the packing bag main body 92 at a rim portion of the upper surface opening portion. An unsealing portion 96 having a weak sealing force is provided at a part of this sealing portion 95. This unsealing portion 96 is arranged at the narrow-width portion 94 of the packing bag main body 92. It is to be noted that an indicating portion 97 such as a label indicating a product name or the like of the insertion support tool 4 accommodated in the packing bag main body 92 is provided in a vacant space of a pedestal 84 of the insertion support tool 4.

Thus, the apparatus having the above-described configuration demonstrates the following effects. That is, in this modification, a mouthpiece member 17 of the insertion support tool 4 can be exposed by unsticking the sealing film 93 from the unsealing portion 96 at the narrow-width portion 94 of the packing bag main body 92. Therefore, an end portion of an inserting portion 93 of a large-intestine endoscope 2 can be inserted from the mouthpiece member 17 of the insertion support tool 4 in a state where the insertion support tool 4 is within the packing bag 83. At this time, since the pedestal 84 of the insertion support tool 4 has a large width, a pressing action can be facilitated during an operation of inserting the end portion of the inserting portion 3 of the large-intestine endoscope 2.

Additionally, in this modification, since any part of the insertion support tool 4 other than the mouthpiece member 17 in the packing bag main body 92 is not exposed, storage until use can be facilitated, thus reducing a risk such as contamination.

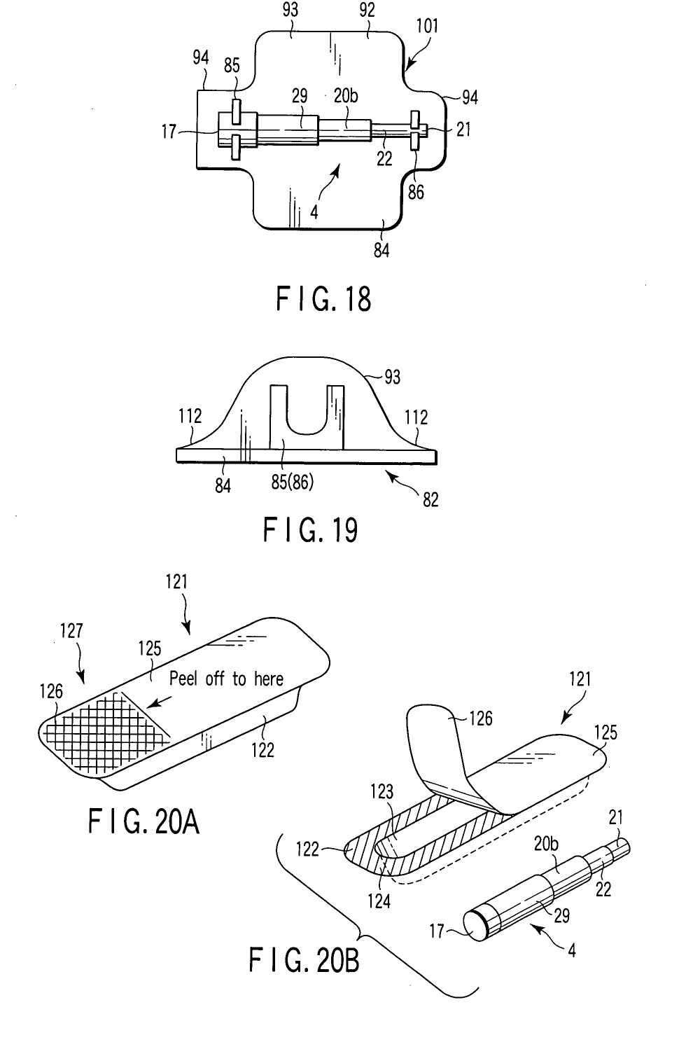

FIG. 18 shows a second modification of the packing bag 83 of the insertion support tool 4 according to the sixth embodiment (see FIGS. 14 to 16). In a packing bag 101 according to this modification, narrow-width portions 94 are formed at both end portions of the packing bag main body 92 of the packing bag 91 according to the first modification (see FIG. 17).

Thus, the apparatus having the above-described configuration demonstrates the following effects. That is, in the packing bag 101 according to this modification, like the packing bag 91 according to the first modification, an end portion of an inserting portion 3 of a large-intestine endoscope 2 is assembled with respect to an insertion support tool 4 in the packing bag main body 92, and then an end side of the insertion support tool 4 is exposed, thereby holding and fixing the inserting portion 3 of the large-intestine endoscope 2. Therefore, the inserting portion 3 of the large-intestine endoscope 2 can be assuredly held and fixed.

FIG. 19 shows a third modification of the packing bag 83 of the insertion support tool 4 according to the sixth embodiment (see FIGS. 14 to 16). In a packing bag 111 according to this modification, an upper side of the pedestal unit 82 according to the sixth embodiment is covered with a sealing film 93, and a sealing portion 112 obtained by welding and fixing a rim part of the sealing film 93 is provided at an outer peripheral part of a pedestal 84. Moreover, the packing bag 111 according to this modification has an effect of facilitating recycling.

It is to be noted that the pedestal 84 and first and second support leg portions 85 and 85 provided thereon may be integrally molded by using, e.g., cardboard in the packing bag 111 according to this modification. Additionally, the sealing film 93 may be formed of a transparent film. In this case, there is an effect that the contents of the packing bag 11 can be directly seen through the transparent sealing film 93.

FIGS. 20A and 20B shows a fourth modification of the packing bag 83 of the insertion support tool 4 according to the sixth embodiment (see FIGS. 14 to 16). In a packing bag 121 according to this modification, an insertion support tool 4 is accommodated in a concave portion 123 of a blister packing 122 formed of a transparent resin material as shown in FIG. 20B. Furthermore, a flange portion 124 is formed at a rim part of the concave portion 123 of the blister packing 122. A rim part of a sealing sheet 125 which closes an upper surface opening portion of the concave portion 123 of the blister packing 122 is attached on this flange portion 124.

A dimension of the concave portion 123 of the blister packing 122 is substantially the same as that of the insertion support tool 4. Moreover, it is preferable for the concave portion 123 of the blister packing 122 to have a depth which allows accommodation of the entire insertion support tool 4. Additionally, a peeling start portion 126 which facilitates peeling from the flange portion 124 of the blister packing 122 is formed on one end portion side of the sealing sheet 125. An indicating portion 127 which indicates a position at which a mouthpiece member 17 of the insertion support tool 4 is exposed is provided to this peeling start portion 126.

Further, in the packing bag 121 according to this modification, since the blister packing 122 is transparent, the insertion support tool 4 accommodated in the concave portion 123 can be visually observed. Furthermore, at the point of use, the mouthpiece member 17 of the insertion support tool 4 alone can be exposed by peeling the peeling start portion 126 on one end portion side of the sealing sheet 125. Therefore, there is an effect that the apparatus is hardly contaminated.

Moreover, since the packing bag 121 according to this modification is configured to accommodate the entire insertion support tool 4 in the concave portion 123 of the blister packing 122, a shape of the insertion support tool 4 can be held, and takeoff can be facilitated.

Additionally, since the indicating portion 127 indicating a position as a guide for exposure of the mouthpiece member 17 of the insertion support tool 4 is provided on one end portion side of the sealing sheet 125, the sealing sheet 125 is not unnecessarily greatly peeled off. Therefore, a risk of contamination is small.

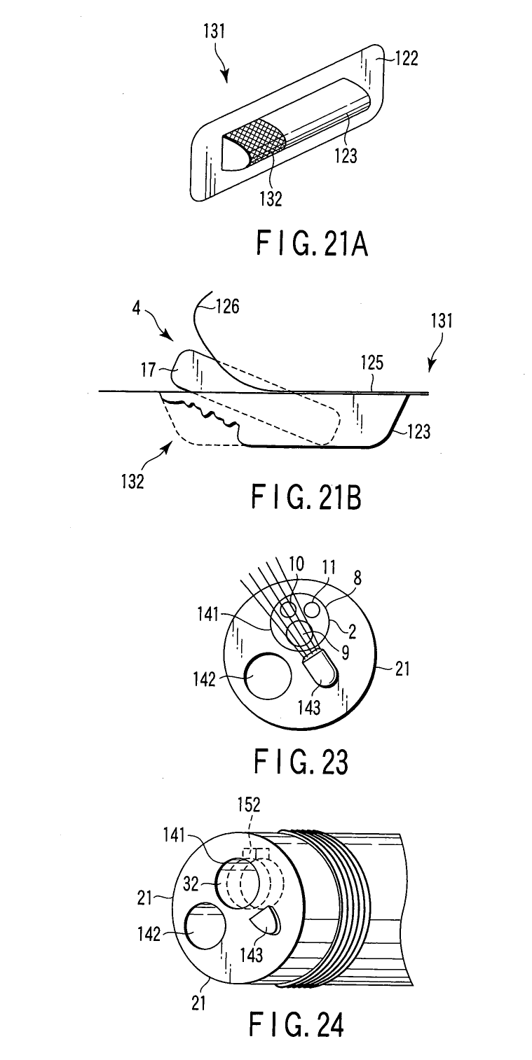

FIGS. 21A and 21B show a fifth modification of the packing bag 93 of the insertion support tool 4 according to the sixth embodiment (see FIGS. 14 to 16). A packing bag 131 according to this modification is obtained by changing the packing bag 121 according to the fourth modification (see FIGS. 20A and 20B) as follows.

That is, in the packing bag 131 according to this modification, as shown in FIG. 21A, a thin-walled portion 132 is formed at one end portion of a concave portion 123 of a blister packing 122. Furthermore, as shown in FIG. 21B, like the packing bag 121 according to the fourth modification, a mouthpiece member 17 of an insertion support tool 4 alone is exposed by peeling a peeling start portion 126 provided on one end portion side of a sealing sheet 125, and the thin-walled portion 132 is held and pushed in this state. As a result, the mouthpiece member 17 of the insertion support tool 4 is exposed to the outside from an upper surface opening portion of the concave portion 123 of the blister packing 122. Therefore, an operation of inserting an end portion of an inserting portion 3 of a large-intestine endoscope 2 into the mouthpiece member 17 of the insertion support tool 4 can be facilitated.

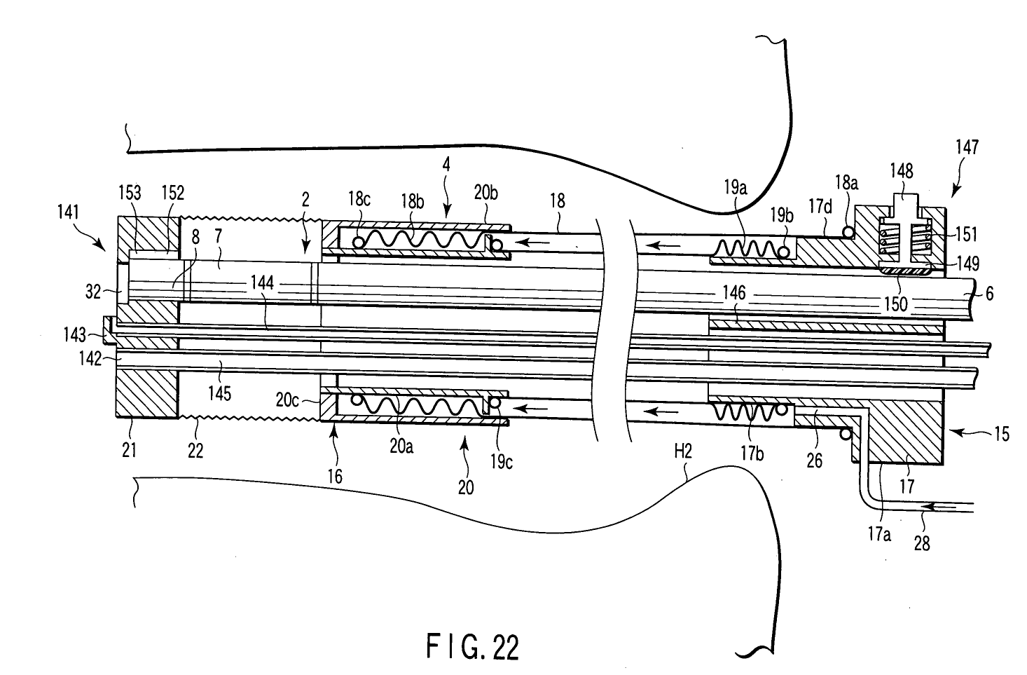

FIGS. 22 to 27B show a seventh embodiment according to the present invention. This embodiment is obtained by changing the configuration of the insertion support tool 4 of the endoscope insertion apparatus 1 according to the first embodiment (see FIGS. 1 to 7A) as follows. It is to be noted that, in FIGS. 22 to 27B, like reference numerals denote parts equal to those depicted in FIGS. 1 to 7A, thereby obviating their explanation.

As shown in FIG. 23, an end member 21 of an end unit 16 of an insertion support tool 4 according to this embodiment has an endoscope attachment portion 141, an end opening portion 142 for suction, and an air supply/water supply nozzle 143. As shown in FIG. 24, an end portion of a suction tube 145 is coupled with the end opening portion 142. An end portion of an air supply/water supply tube 144 is coupled with the air supply/water supply nozzle 143. The air supply/water supply nozzle 143 is fixed in a state where it emits a fluid toward a cover glass 32 of the endoscope attachment portion 141. Additionally, a key groove 152 is formed at a part of an endoscope attachment hole 31 of the end member 21 in a circumferential direction. This key groove 152 is extended in an inserting direction of an inserting portion 3 of a large-intestine endoscope 2.

Further, as shown in FIG. 25, a key 153 which is releasably engaged with the key groove 152 is provided to protrude from an end hard portion 8 of the large-intestine endoscope 2. Furthermore, a rubber ring 154 is attached on an outer peripheral surface of the end hard portion 8. Moreover, at the time of an operation of attaching the insertion support tool 4 with respect to the inserting portion 3 of the large-intestine endoscope 2, the key 153 of the end hard portion 8 and the key groove 152 of the end member 21 are positioned and engaged. As a result, when the insertion support tool 4 is attached to the inserting portion 3 of the large-intestine endoscope 2, the end hard portion 8 can be positioned in the circumferential direction in the endoscope attachment hole 31 of the end member 21 by engagement established between the key 153 of the end hard portion 8 and the key groove 152 of the end member 21. Therefore, as shown in FIG. 23, a spouting fluid (washing water) from the air supply/water supply nozzle 143 can be appropriately emitted toward an observation window 9 of the end hard portion 8. Additionally, when the end hard portion 8 is inserted into the endoscope attachment hole 31 of the end member 21, the end hard portion 8 is fastened and fitted in the endoscope attachment hole 31 of the end member 21 by the rubber ring 154 of the end hard portion 8 so that engagement between both the members cannot be released.

Further, as shown in FIG. 26, a tabular support member 146 which partitions a rear end opening portion in the vertical direction is arranged on a mouthpiece member 17 of the insertion support tool 4. In FIG. 26, a first interior space 146a is formed on an upper side of this support member 146 and a second interior space 146b is formed on a lower side of the same, respectively. Furthermore, the suction tube 145 and the air supply/water supply tube 144 are inserted into the first interior space 146a. The inserting portion 3 of the large-intestine endoscope 2 is inserted into the second interior space 146b.

Moreover, a circumferential wall portion on the second interior space 146b side has an endoscope pressing portion 147. This endoscope pressing portion 147 has an operation button 148 as shown in FIG. 27B. An outer end portion of this operation button 148 protrudes toward the outside of the mouthpiece member 17. Additionally, a shaft portion 148a of the operation button 148 protrudes toward the inside of the mouthpiece member 17. A pushing member 149 is fixed at an inner end portion of this shaft portion 148a. As shown in FIG. 27A, this pushing member 149 is formed into an arc shape corresponding to a flexible tube portion 6 of the inserting portion 3 of the large-intestine endoscope 2. Further, a nonslip member 150 formed of a rubber member is fixed on an inner surface of the pushing member 149.

Furthermore, a return spring 151 formed of a coil spring is attached to the shaft portion 148a of the operation button 148. This return spring 151 gives an impetus to the operation button 148 in a direction along which this button protrudes toward the outside of the mouthpiece member 17. Moreover, as indicated by an arrow in FIG. 27B, pushing the operation button 148 against the impetus of the return spring 151 causes the flexible tube portion 6 of the inserting portion 3 of the large-intestine endoscope 2 to be held between the pushing member 149 and the support member 146, thereby generating a frictional force. As a result, the inserting portion 3 of the large-intestine endoscope 2 can be releasably engaged with and fixed to the base unit 15.

Thus, the apparatus having the above-described configuration demonstrates the following effects. That is, in this embodiment, the inserting portion 3 of the large-intestine endoscope 2 can be moved back by pulling the base unit 15 of the insertions support tool 4 toward the front side (a direction opposite to the inserting direction of the inserting portion 3 of the large-intestine endoscope 2) while pushing the operation button 148 against the impetus of the return spring 151. At this time, the inserting portion 3 of the large-intestine endoscope 2 can be moved back in a state where a length between the base unit 15 of the insertion support tool 4 and the end member 21 is fixed. Therefore, during the operation of moving back the inserting portion 3 of the large-intestine endoscope 2, an outer sheath 18 and an inner sheath 19 of the insertion support tool 4 are not folded in a disorderly manner, or an already folded part is not unfolded.

Additionally, the mouthpiece member 17 of the insertion support tool 4 is partitioned into the first interior space 146a and the second interior space 146b by the tabular support member 146. Further, since the suction tube 145 and the air supply/water supply tube 144 are inserted into the first interior space 146a and the inserting portion 3 of the large-intestine endoscope 2 is inserted into the second interior space 146b, the suction tube 145 or the air supply/water supply tube 144 in the insertion support tool 4 is protected by the support member 146. As a result, the suction tube 145 or the air supply/water supply tube 144 in the insertion support tool 4 is not damaged during the operation of moving back the inserting portion 3 of the large-intestine endoscope 2.

The endoscope pressing portion 147 according to this embodiment has a configuration in which a force of pushing the operation button 148 is directly transmitted as a pushing force of the inserting portion 3 of the large-intestine endoscope 2, i.e., a locking force when locking with the flexible tube portion 6 of the inserting portion 3 of the large-intestine endoscope 2 being held between the pushing member 149 and the support member 146. Therefore, a large frictional force can be generated with a compact mechanism.

Furthermore, since the nonslip member 150 formed of the rubber member is fixed on the inner surface of the pushing member 149, a large frictional force can be generated, and it is possible to perform an assured operation of locking with the flexible tube portion 6 of the inserting portion 3 of the large-intestine endoscope 2 being held. Here, as shown in FIG. 27A, since the pushing member 149 is formed into the arc shape corresponding to the flexible tube portion 6 of the inserting portion 3 of the large-intestine endoscope 2, thus generating a larger frictional force.

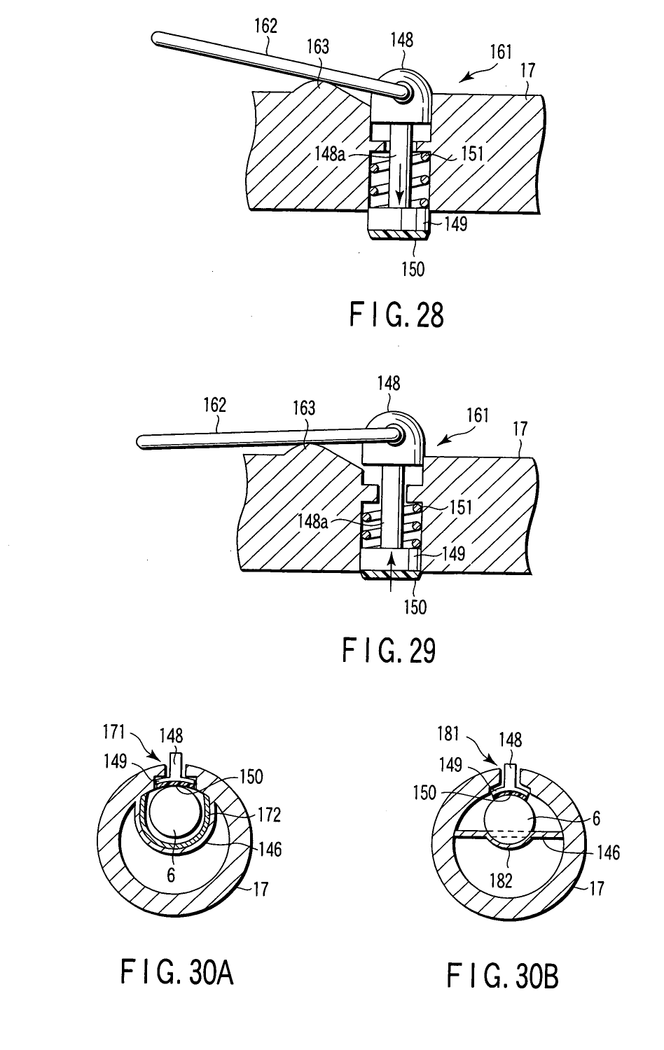

FIGS. 28 and 29 show a first modification of the endoscope pressing portion 147 of the insertion support tool 4 of the endoscope insertion portion 1 according to the seventh embodiment (see FIGS. 22 to 27B). In an endoscope pressing portion 161 according to this modification, a return spring 151 of a shaft portion 148a of an operation button 148 is provided in a state where an impetus functions in a direction along which a pushing member 149 is pushed toward the inside of a mouthpiece member 17. It is to be noted that FIG. 28 shows a state where the operation button 148 is pushed and FIG. 29 shows a state where pushing of the operation button 148 is released.

Additionally, one end portion of an operation lever 162 which releases a locked state of an inserting portion 3 of a large-intestine endoscope 2 by the endoscope pressing portion 161 is coupled with a head portion of the operation button 148 in such a manner that this end portion can swivel. As shown in FIG. 29, when this operation lever 162 is moved down toward a protruding receiving portion (a supporting point) 163 side of a mouthpiece member 17, the pushing member 149 is moved away from a flexible tube portion 6 of the inserting portion 3 of the large-intestine endoscope 2. As a result, the inserting portion 3 of the large-intestine endoscope 2 can move forward and backward.

Thus, the apparatus having the above-described configuration demonstrates the following effects. That is, the endoscope pressing portion 161 according to this modification has a configuration in which the pushing member 149 is moved away from the flexible tube portion 6 of the inserting portion 3 of the large-intestine endoscope 2 when the operation lever 162 is moved down toward the protruding receiving portion 163 side of the mouthpiece member 17. Therefore, when the mouthpiece member 17 is grasped by hand, it is possible to avoid a pushing operation of the operation button 148 which causes the endoscope pressing portion 161 to operate.

Further, FIG. 30A shows a second modification of the endoscope pressing portion 147 of the insertion support tool 4 according to the seventh embodiment (see FIGS. 22 to 27B). In an endoscope pressing portion 171 according to this modification, a support member 146 of a mouthpiece member 17 is curved along an outer shape of an inserting portion 3 of a large-intestine endoscope 2 and a nonslip 172 formed of a rubber member is provided on an inner peripheral surface of the support member 146 having the curved shape.

Accordingly, the apparatus having the above-described configuration demonstrates the following effects. That is, in the endoscope pressing portion 171 according to this modification, a contact area of the support member 146 having the curved shape and the inserting portion 3 of the large-intestine endoscope 2 is large, and hence a large frictional force can be obtained.

Furthermore, FIG. 30B shows a third modification of the endoscope pressing portion 147 of the insertion support tool 4 according to the seventh embodiment (see FIGS. 22 to 27B). In an endoscope pressing portion 181 according to this modification, a support member 146 of a mouthpiece member 17 is formed of a material having hardness which allows appropriate transformation. Here, the hardness of the support member 146 which allows appropriate transformation is a hardness which allows transformation without damaging a suction tube 145 or an air supply/water supply tube 144 in a first interior space 146a when an operation button 148 is pushed and a pushing member 149 thereby pushes a flexible tube portion 6 of an inserting portion 3 of a large-intestine endoscope 2.

Thus, the apparatus having the above-described configuration demonstrates the following effects. That is, in the endoscope pressing portion 181 according to this modification, when the operation button 148 is pushed, the support member 146 appropriately transforms in accordance with an outer shape of the inserting portion 3 of the large-intestine endoscope 2. Therefore, a contact area of a transformed portion 182 having a curved shape of the support member 146 and the inserting portion 3 of the large-intestine endoscope 2 is large, thereby obtaining a large frictional force.

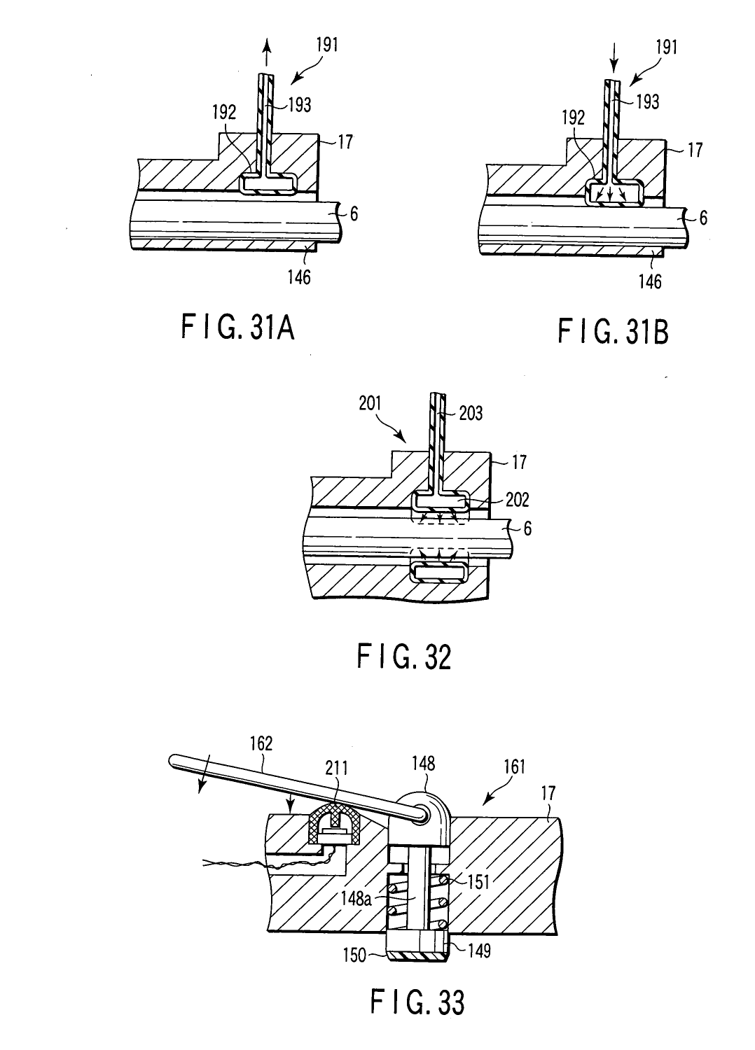

FIGS. 31A and 31B show an eighth embodiment according to the present invention. This embodiment is obtained by changing the configuration of the endoscope pressing portion 147 of the inserting support tool 4 according to the seventh embodiment (see FIGS. 22 to 27B) as follows.

That is, in an endoscope pressing portion 191 according to this embodiment, an inflatable balloon 192 is provided in place of the pushing member 149 of the operation button 148 according to the seventh embodiment as shown in FIG. 31A. One end portion of an air supply tube 193 is connected with this balloon 192. The other end portion of this air supply tube 193 is connected with an air supply source of a non-illustrated compressor or the like. Further, when air or the like is supplied into the balloon 192, as shown in FIG. 31B, the balloon 192 inflates. At this time, a flexible tube portion 6 of an inserting portion 3 of a large-intestine endoscope 2 is held and locked between the balloon 192 and a support member 146.

Thus, the apparatus having the above-described configuration demonstrates the following effects. That is, in the endoscope pressing portion 191 according to this embodiment, since the balloon 192 is provided in place of the pushing member 149 of the operation button 148 according to the seventh embodiment, the configuration is compact and a weight is reduced. Therefore, the entire insertion support tool 4 can be reduced in size and weight, thereby lessening fatigue during endoscopy.

FIG. 32 shows a ninth embodiment according to the present invention. This embodiment is obtained by changing the configuration of the endoscope pressing portion 147 of the insertion support tool 4 of the seventh embodiment (see FIGS. 22 to 27B) as follows.

That is, in an endoscope pressing portion 201 according to this embodiment, a donut-like balloon 202 is provided in place of the balloon 192 according to the eighth embodiment (see FIGS. 31A and 31B). One end portion of an air supply tube 203 is connected with this balloon 202. The other end portion of this air supply tube 203 is connected with an air supply source of a non-illustrated compressor or the like. Moreover, when air or the like is supplied into the balloon 202, the balloon 202 inflates as indicated by dotted lines in FIG. 32. At this time, a flexible tube portion 6 of an inserting portion 3 of a large-intestine endoscope 2 is compressed and locked from an entire circumference of the balloon 202.

Thus, the apparatus having the above-described configuration demonstrates the following effects. That is, in the endoscope pressing portion 201 according to this embodiment, since the donut-like balloon 202 is provided, the flexible tube portion 6 of the inserting portion 3 of the large-intestine endoscope 2 can be compressed and locked from the entire circumference of the balloon 202. Therefore, a large frictional force can be obtained.

FIG. 33 shows a 10th embodiment according to the present invention. This embodiment is obtained by changing the configuration of the endoscope pressing portion 147 of the insertion support tool 4 of the seventh embodiment (see FIGS. 22 to 27B) as follows.

That is, in this embodiment, for example, a driving switch 211 of a compressor (or a switch of a valve which controls air from the compressor) which supplies a fluid to an external sheath 14 of an insertion support tool 4 is provided at a position of the receiving portion (a supporting point) 163 of the operation lever 162 in the endoscope pressing portion 161 according to the first modification (see FIGS. 28 and 29) of the seventh embodiment.

Further, in this embodiment, when an operation lever 162 of an endoscope pressing portion 161 is moved down toward a receiving portion 163 side, a locked state of a pushing member 149 of an operation button 148 is released and, at the same time, the driving switch 211 is turned on, thereby supplying a fluid to the external sheath 14.

Thus, the apparatus having the above-described configuration demonstrates the following effects. That is, in this embodiment, two operations, i.e., releasing the locked state of the pushing member 149 of the operation button 148 and supplying a fluid to the external sheath 14 can be performed by one operation of moving down the operation lever 162 of the endoscope pressing portion 161 toward the receiving portion 163 side.

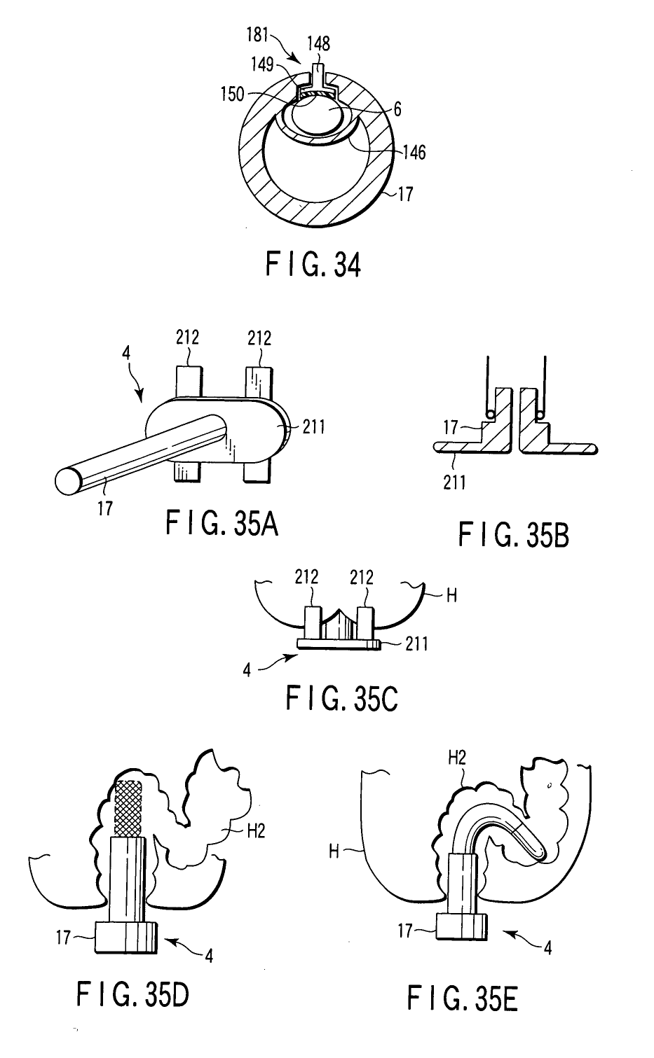

FIG. 34 shows an 11th embodiment according to the present invention. This embodiment is obtained by changing the configuration of the endoscope insertion apparatus 1 of the seventh embodiment (see FIGS. 22 to 27B) as follows.

That is, in this embodiment, a lateral cross-sectional shape of an inserting portion 3 of a large-intestine endoscope 2 is formed into an elliptic shape. Moreover, a lateral cross-sectional shape of a second interior space 146b of a support member 146 is formed into an elliptic shape corresponding to the elliptic shape of the inserting portion 3 of the large-intestine endoscope 2. As a result, when the inserting portion 3 of the large-intestine endoscope 2 or a mouthpiece member 17 of the insertion support tool 4 is twisted in a periaxial direction with the inserting portion 3 of the large-intestine endoscope 2 being inserted in the second interior space 146b, both the inserting portion 3 of the large-intestine endoscope 2 and the insertion support tool 4 can be prevented from being freely rotated.

Thus, the apparatus having the above-described configuration demonstrates the following effects. That is, in this embodiment, when the inserting portion 3 of the large-intestine endoscope 2 or the mouthpiece member 17 of the insertion support tool 4 is twisted in the periaxial direction, follow-up properties with respect to rotation are good, and a treatment can be excellently performed.

FIGS. 35A to 35E show a 12th embodiment according to the present invention. This embodiment is obtained by changing the configuration of the primary part of the insertion support tool 4 of the endoscope insertion apparatus 1 of the first embodiment (see FIGS. 1 to 7A) as follows.

That is, in this embodiment, as shown in FIG. 35A, a flange portion 211 is provided at a rear end surface of a mouthpiece member 17 of an insertion support tool 4. As depicted in FIG. 35B, this flange portion 211 is integrally formed with the mouthpiece member 17. Further, as shown in FIG. 35C, when the insertion support tool 4 is inserted into an anus, the flange portion 211 of the mouthpiece member 17 is fixed on a body wall such as the buttocks of a patient by an adhesive tape 212 or the like.

Thus, the apparatus having the above-described configuration demonstrates the following effects. That is, in this embodiment, when the insertion support tool 4 is inserted into the anus, the flange portion 211 of the mouthpiece member 17 can be fixed on the body wall such as buttocks of a patient by the adhesive tape 212 or the like. Therefore, for example, as shown in FIG. 35D, when an extending force is applied to a rear side rather than a front side in a state where an end of the insertion support tool 4 is in contact with a bent part in a large intestine H2, the insertion support tool 4 can be prevented from coming out of the anus. On the contrary, for example, in a case where the insertion support tool 4 is pulled into the anus when the patient moves, movement of the insertion support tool 4 can be suppressed. As a result, an operator or a helper does not have to hold the mouthpiece member 17 during the operation of the insertion support tool 4, thereby making the task less strenuous.

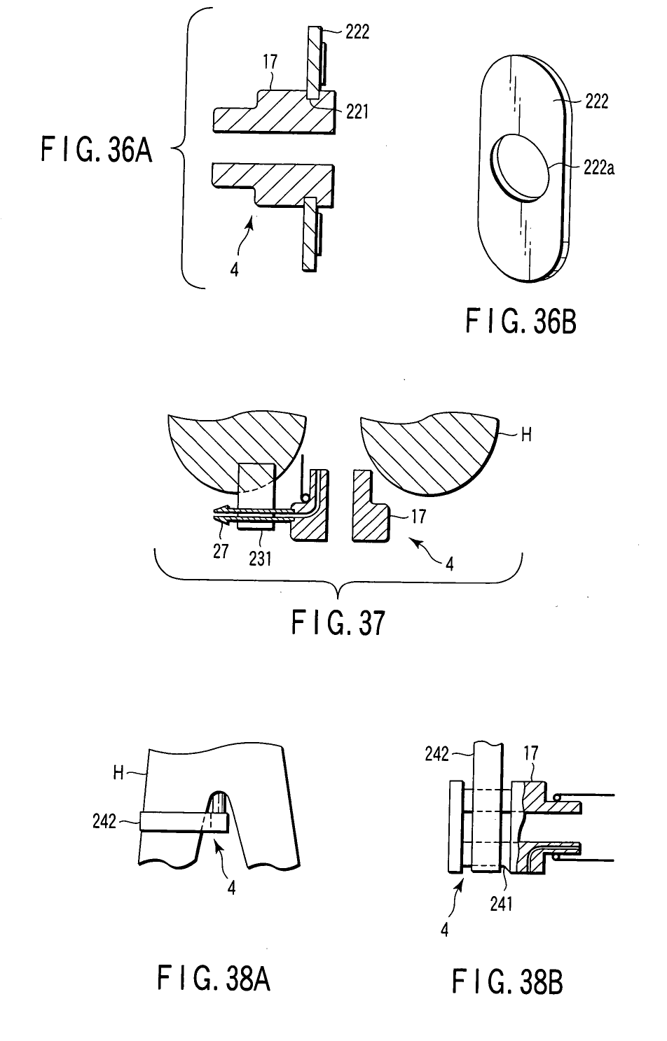

FIGS. 36A and 36B show a modification of the flange portion 211 of the insertion support tool 4 of the endoscope insertion apparatus 1 according to the 12th embodiment (see FIGS. 35A to 35E). In this modification, a flange attachment groove 221 is provided on an outer peripheral surface of a rear end portion of a mouthpiece member 17 of an insertion support tool 4 as shown in FIG. 36A. A flange constituent member 222, which is different from the mouthpiece member 17, is detachably attached in this flange attachment groove 221. As depicted in FIG. 36B, this flange constituent member 222 is formed of an elastic member, and a circular fitting hole 222a is formed at an axial center portion thereof. Furthermore, the fitting hole 222a can be fitted on the flange attachment groove 221 of the mouthpiece member 17 by elastically deforming the flange constituent member 222.

Thus, the apparatus having the above-described configuration demonstrates the following effects. That is, in this modification, since the flange constituent member 222 is detachably attached in the flange attachment groove 221 of the mouthpiece member 17, the flange constituent member 222 can be readily taken out from the flange attachment groove 221 of the mouthpiece member 17 when it is unnecessary. Therefore, the flange constituent member 222 of the mouthpiece member 17 is not an obstacle at the time of transport or disposal.

FIG. 37 shows an insertion support tool 4 according to a 13th embodiment of the present invention. This embodiment is obtained by changing the configuration of the primary part of the insertion support tool 4 in the endoscope insertion apparatus 1 of the first embodiment (see FIGS. 1 to 7A) as follows.

That is, in this embodiment, an air supply mouthpiece 27 of a mouthpiece member 17 of an insertion support tool 4 is extended long in a lateral direction. Moreover, when the insertion support tool 4 is inserted into an anus, the air supply mouthpiece 27 is fixed on a body wall such as the buttocks of a patient by an adhesive tape 231.

Thus, the apparatus having the above-described configuration demonstrates the following effects. That is, in this embodiment, the air supply mouthpiece 27 can be used in place of the flange portion 211 of the insertion support tool 4 according to the 12th embodiment (see FIGS. 35A to 35E). Therefore, the number of components is not increased, which is efficient.

FIGS. 38A and 38B show an insertion support tool 4 according to a 14th embodiment of the present invention. This embodiment is obtained by changing the configuration of the primary part of the insertion support tool 4 in the endoscope insertion apparatus 1 of the first embodiment (see FIGS. 1 to 7A) as follows.

That is, in this embodiment, a belt winding groove 241 is provided on an outer peripheral surface of a mouthpiece member 17 of an insertion support tool 4. Additionally, when the insertion support tool 4 is inserted into an anus, as shown in FIG. 38A, the insertion support tool 4 is fixed to a patient H by winding a fixing belt 242 between the belt winding groove 241 of the mouthpiece member 17 and a leg of a patient H. As the fixing belt 242, it is possible to use a buckle type belt, a Velcro strap (a registered trademark), a rubber band and others.

Thus, the apparatus having the above-described configuration demonstrates the following effects. That is, in this embodiment, since the fixing belt 242 is wound between the belt winding groove 241 of the mouthpiece member 17 and the leg of the patient H, fixation of the insertion support tool 4 is assured.

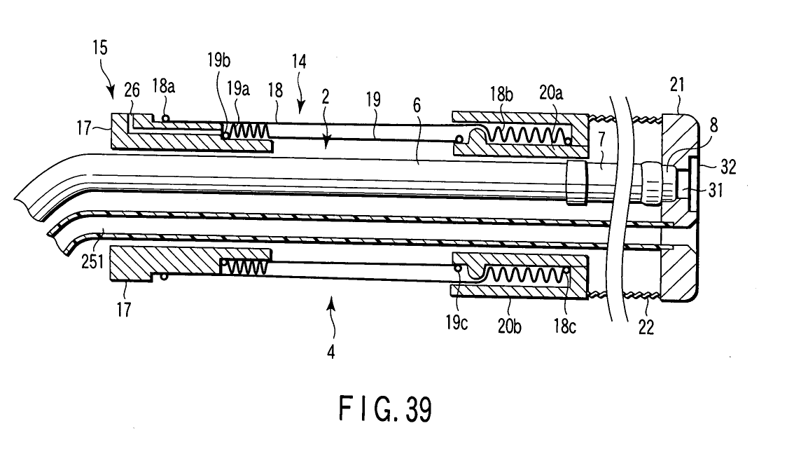

FIGS. 39 to 41C show a 15th embodiment according to the present invention. This embodiment is obtained by changing the configuration of the primary part of the insertion support tool 4 in the endoscope insertion apparatus 1 of the first embodiment (see FIGS. 1 to 7A) as follows.

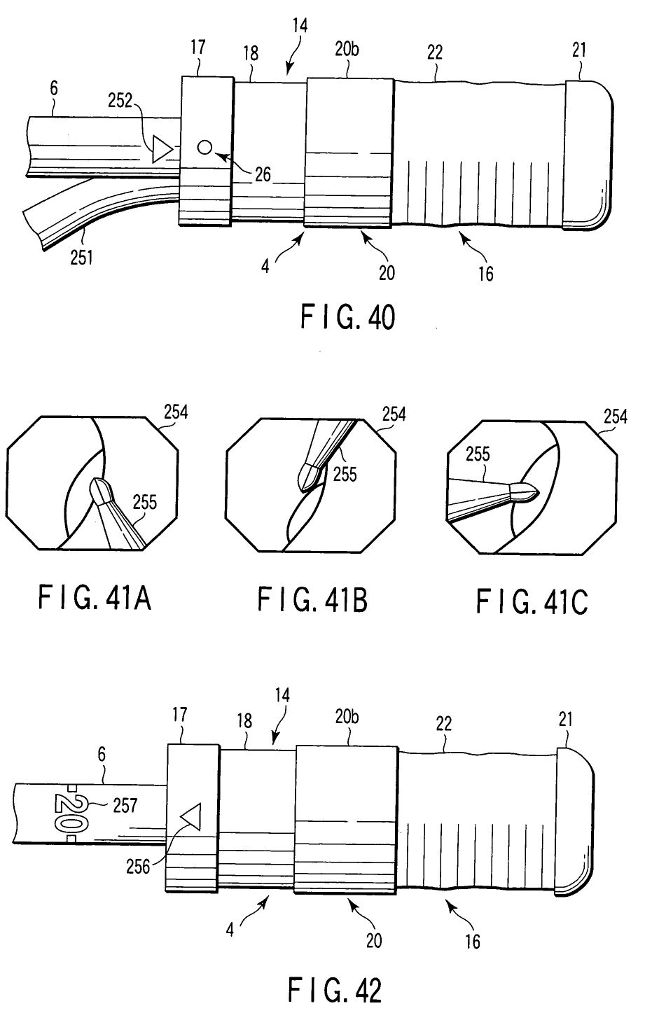

As shown in FIG. 39, an insertion support tool 4 according to this embodiment has an endoscope attachment hole 31 and a forceps channel 251 in an end member 21. Furthermore, as shown in FIG. 40, a positioning index 252 for positioning with a mouthpiece member 17 of a base unit 15 is provided to an inserting portion 3 of a large-intestine endoscope 2.

A function of the above-mentioned configuration will now be described. In this embodiment, at the time of an operation of assembling the insertion support tool 4 to an end portion of the inserting portion 3 of the large-intestine endoscope 2, the positioning index 252 of the insertion portion 3 of the large-intestine endoscope 2 is positioned with the mouthpiece member 17 of the base unit 15. As a result, the insertion support tool 4 can be assembled with respect to the end portion of the inserting portion 3 of the large-intestine endoscope 2 in a state where a rotating position of the inserting portion 3 of the large-intestine endoscope 2 in a periaxial direction is correctly positioned with respect to the insertion support tool 4. Therefore, a position of the forceps channel 251 with respect to an observation image of the large-intestine endoscope 2 can be set to a fixed relationship. As a result, a direction along which an instrument appears in an observation image of the large-intestine endoscope 2 can be fixed.

It is to be noted that FIGS. 41A to 41C show display states of an instrument 255 in an observation image of the large-intestine endoscope 2. For example, FIG. 41A shows a state where the instrument 255 appears from a lower side of a screen 254, FIG. 41B shows a state where the instrument 255 appears from an upper side of the screen 254, and FIG. 41C shows a state where the instrument 255 appears from a left side of the screen 254, respectively.

Thus, the apparatus having the above-described configuration demonstrates the following effects. That is, in this embodiment, at the time of an operation of assembling the insertion support tool 4 at the end portion of the inserting portion 3 of the large-intestine endoscope 2, the positioning index 252 of the inserting portion 3 of the large-intestine endoscope 2 is positioned with the mouthpiece member 17 of the base unit 15. As a result, the insertion support tool 4 can be assembled with respect to the end portion of the inserting portion 3 of the large-intestine endoscope 2 in a state where a rotating position of the inserting portion 3 of the large-intestine endoscope 2 in a periaxial direction is correctly positioned with respect to the insertion support tool 4. As a result, a display state of the instrument 255 in an observation image of the large-intestine endoscope 2 can be set to a state which facilitates an operation by a user, e.g., one of FIGS. 41A to 41C. At this time, since a direction in the observation image of the large-intestine endoscope 2 along which the forceps channel 251 is arranged can be assuredly recognized, an operation of performing suction through the forceps channel 251 can be facilitated.

FIG. 42 shows a 16th embodiment according to the present invention. This embodiment is obtained by changing the configuration of the endoscope insertion apparatus 1 of the 15th embodiment (see FIGS. 39 to 41C) as follows.

That is, in this embodiment, a positioning index 256 is provided on a mouthpiece member 17 of an insertion support tool 4. Moreover, at the time of an operation of assembling the insertion support tool 4 at an end portion of an inserting portion 3 of the large-intestine endoscope 2, an insertion length index 257 of the inserting portion 3 of the large-intestine endoscope 2 is positioned with the positioning index 256 of the mouthpiece member 17.

Thus, also in the apparatus having the above-described configuration, the insertion support tool 4 can be assembled at the end portion of the inserting portion 3 of the large-intestine endoscope 2 in a state where a rotating position of the inserting portion 3 of the large-intestine endoscope 2 in a periaxial direction is correctly positioned with respect to the insertion support tool 4 like the 15th embodiment. As a result, a display state of an instrument 255 in an observation image of the large-intestine endoscope 2 can be set to a state which facilitates an operation by a user, e.g., one of FIGS. 41A to 41C.

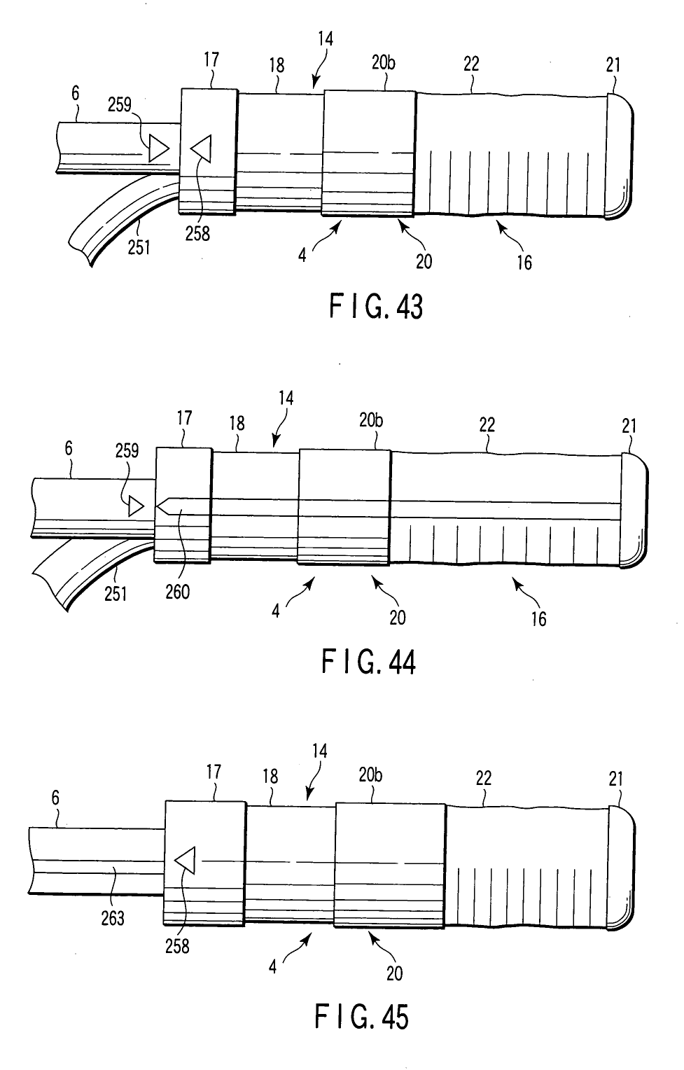

FIG. 43 shows a 17th embodiment according to the present invention. This embodiment is obtained by changing the configuration of the endoscope insertion apparatus 1 of the 15th embodiment (see FIGS. 39 to 41C) as follows.

That is, in this embodiment, a positioning index 258 is provided on a mouthpiece member 17 of an insertion support tool 4. Additionally, a positioning index 259 is likewise provided on an inserting portion 3 of a large-intestine endoscope 2. Further, at the time of an operation of assembling the inserting support tool 4 at an end portion of the inserting portion 3 of the large-intestine endoscope 2, the positioning index 259 on the inserting portion 3 of the large-intestine endoscope 2 is positioned with respect to the positioning index 258 on the mouthpiece member 17.

Thus, also in the apparatus having the above-described configuration, the insertion support tool 4 can be assembled at the end portion of the inserting portion 3 of the large-intestine endoscope 2 in a state where a rotating position of the inserting portion 3 of the large-intestine endoscope 2 in a periaxial direction is correctly positioned with respect to the insertion support tool 4 like the 15th embodiment. As a result, a display state of an instrument 255 in an observation image of the large-intestine endoscope 2 can be set to a state which facilitates an operation by a user, e.g., one of FIGS. 41A to 41C.

FIG. 44 shows an 18th embodiment according to the present invention. This embodiment is obtained by changing the configuration of the endoscope insertion apparatus 1 according to the 17th embodiment (see FIG. 43) as follows. That is, in this embodiment, a positioning index 260 linearly extended from a mouthpiece member 17 of an insertion support tool 4 to an end member 21 is provided. Furthermore, at the time of an operation of assembling the insertion support tool 4 at an end portion of an inserting portion 3 of a large-intestine endoscope 2, a positioning index 259 on the inserting portion 3 of the large-intestine endoscope 2 is positioned with the positioning index 260 on the insertion support tool 4.

Here, when distortion is generated between the mouthpiece member 17 of the insertion support tool 4 and the end member 21, a forceps channel 251 is displaced in an observation image of the large-intestine endoscope 2 even if the positioning index 260 and the positioning index 259 on the inserting portion 3 of the large-intestine endoscope 2 are positioned at the position of the mouthpiece member 17 of the insertion support tool 4. Thus, in the apparatus having the above-described configuration, since a positional relationship with the positioning index 259 on the inserting portion 3 of the large-intestine endoscope 2 can be confirmed at the position of the end member 21 of the insertion support tool 4, the insertion support tool 4 can be assembled at the end portion of the inserting portion 3 of the large-intestine endoscope 2 in a state where a rotating position of the inserting portion 3 of the large-intestine endoscope 2 in a periaxial direction is correctly positioned with respect to the insertion support tool 4. As a result, a display state of an instrument 255 in an observation image of the large-intestine endoscope 2 can be set to a state which facilitates an operation by a user, e.g., one of FIGS. 41A to 41C.

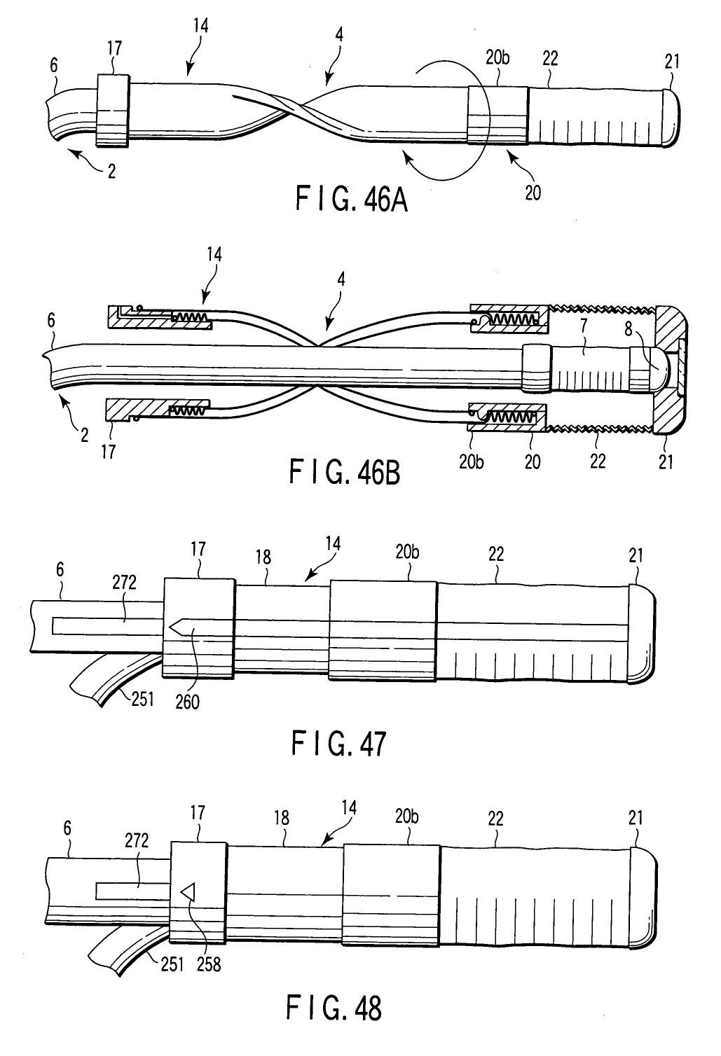

Moreover, FIGS. 45, 46A and 46B show a 19th embodiment according to the present invention. This embodiment is obtained by changing the configuration of the endoscope insertion apparatus 1 according to the 17th embodiment (see FIG. 43) as follows. That is, in this embodiment, a positioning index 263 linearly extended over an entire effective length of an inserting portion 3 is provided on the inserting portion 3 of a large-intestine endoscope 2. Additionally, at the time of an operation of assembling an insertion support tool 4 at an end portion of the inserting portion 3 of the large-intestine endoscope 2, the positioning index 263 on the inserting portion 3 of the large-intestine endoscope 2 is positioned with a positioning index 258 on the insertion support tool 4.

Here, when distortion is generated between a mouthpiece member 17 of the insertion support tool 4 and an end member 21 as shown in FIGS. 46A and 46B during an operation of extending an external sheath 14 of the insertion support tool 4, the external sheath 14 is crushed at a distorted part, and air used to extend the external sheath 14 is hardly supplied. Thus, in the apparatus having the above-described configuration, extending the external sheath 14 while positioning the positioning index 263 on the inserting portion 3 of the large-intestine endoscope 2 and the positioning index 258 on the insertion support tool 4 can avoid an extension defect caused due to distortion of the external sheath 14 during the operation of extending the external sheath 14.

FIG. 47 shows a 20th embodiment according to the present invention. This embodiment is obtained by changing the configuration of the endoscope insertion apparatus 1 according to the 18th embodiment (see FIG. 44) as follows. That is, in this embodiment, a positioning index 260 linearly extended from a mouthpiece member 17 of an insertion support tool 4 to an end member 21 is provided. Furthermore, a linear positioning index 272 whose length is larger than that covered with the contracted insertion support tool 4 is provided on the inserting portion 3 of the large-intestine endoscope 2.

Moreover, at the time of an operation of assembling the insertion support tool 4 at an end portion of the inserting portion 3 of the large-intestine endoscope 2, the positioning index 260 on the insertion support tool 4 is used to confirm that an external sheath 14 is not distorted. Additionally, the positioning index 260 on the insertion support tool 4 is positioned at an end of the positioning index 272 on the inserting portion 3 of the large-intestine endoscope 2, and insertion and attachment are performed in such a manner that a set position is not shifted.

Thus, in the apparatus having the above-described configuration, the insertion support tool 4 can be attached without distortion of the external sheath 14 at the time of an operation of assembling the insertion support tool 4 at the end portion of the inserting portion 3 of the large-intestine endoscope 2. Further, during the assembling operation, this operation can be carried out without distorting the external sheath 14.

FIG. 48 shows a 21st embodiment according to the present invention. This embodiment is obtained by changing the configuration of the endoscope insertion apparatus 1 according to the 17th embodiment (see FIG. 43) as follows. That is, in this embodiment, a linear positioning index 272 whose length is larger than a length covered with a contracted insertion support tool 4 is provided on an inserting portion 3 of a large-intestine endoscope 2.

Moreover, at the time of an operation of assembling the insertion support tool 4 at an end portion of the inserting portion 3 of the large-intestine endoscope 2, a positioning index 258 on the insertion support tool 4 is positioned at an end of the positioning index 272 on the inserting portion 3 of the large-intestine endoscope 2, and insertion and attachment are carried out in such a manner that the set position is not shifted.

Thus, in the apparatus having the above-described configuration, at the time of an operation of assembling the insertion support tool 4 at the end portion of the inserting portion 3 of the large-intestine endoscope 2, this operation can be performed without distorting an external sheath 14.

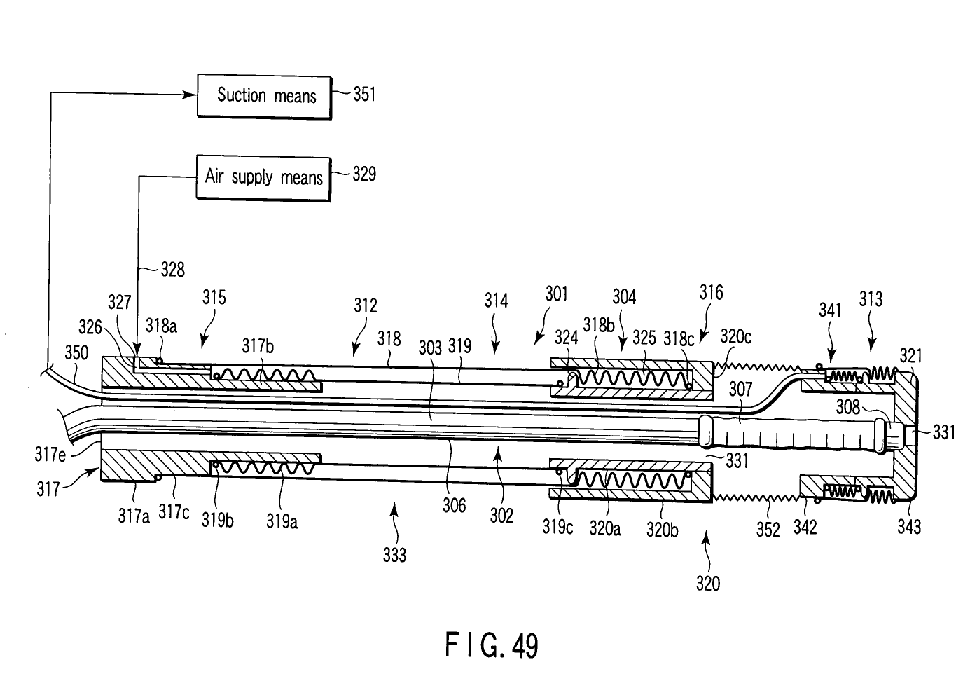

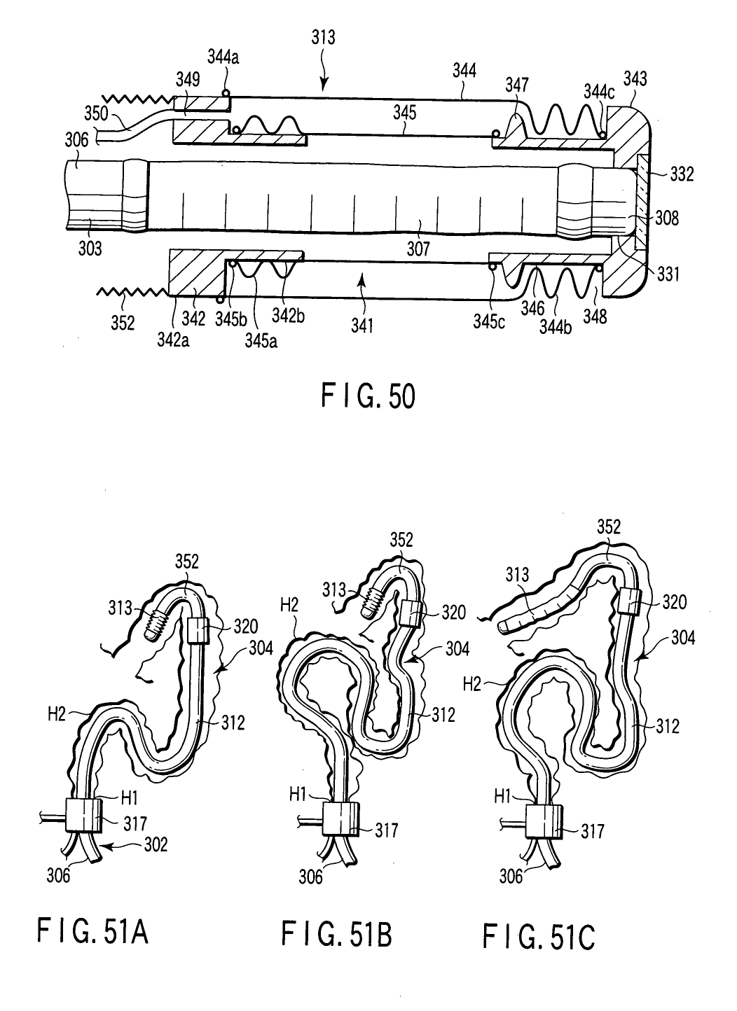

FIGS. 49 to 51C show a 22nd embodiment according to the present invention. FIG. 49 shows an endoscope insertion apparatus 301 which is an insertion support tool for a medical instrument according to this embodiment. The endoscope insertion apparatus 301 has an insertion support tool 304 which is detachably attached at an end portion of an inserting portion 303 of a large-intestine endoscope 302.

The large-intestine endoscope 302 has a large-diameter operating portion at a base end portion of the elongated shaft-shaped inserting portion 303 which is inserted into a body. The inserting portion 303 has an elongated flexible tube portion 306, a curving portion 307 which can curve in four directions, e.g., up, down, right and left directions, and an end hard portion 308. Furthermore, a base end portion of the flexible tube portion 306 is coupled with the operating portion. An end portion of a non-illustrated curving operation wire is coupled with the curving portion 307.

An end surface of the end hard portion 308 has at least one observation window and an illumination window. An objective lens is arranged behind a cover glass of the observation window. An imaging element such as a CCD is arranged at an image forming position of this objective lens. One end portion of a signal cable is connected with this imaging element. An end portion of a light guide fiber bundle is arranged behind a cover glass of the illumination window.

Moreover, the signal cable of the imaging element, the light guide fiber bundle, the curving operation wire and others are extended toward an operating portion side through the inside of the inserting portion 303. Additionally, the signal cable, of the imaging element, the light guide fiber bundle, the curving operation wire and others are packed in the inserting portion 303 as built-in matters.

A curving operation knob which operates the curving portion 307 is arranged on the operating portion. A central shaft of the curving operation knob is coupled with a curving operation mechanism built into the operating portion. A base end portion of the curving operation wire is coupled with this curving operation mechanism. Further, the curving operation wire is towed via the curving operation mechanism by a rotating operation of the curving operation knob so that the curving portion 307 is remotely operated in an operating direction of the curving operation knob.

Furthermore, one end portion of a universal cord is coupled with the operating portion. A light connector and an electric signal connector are provided at the other end portion of this universal cord. The light connector is connected with a light source device and the electric signal connector is connected with a video processor, respectively. Here, the signal cable or the light guide fiber bundle is inserted into the universal cord from the operating portion. Moreover, the signal cable is connected with the electric signal connector.

Additionally, the other end portion of the light guide fiber bundle is connected with the light connector. Further, illumination light emitted from a light source lamp of the light source device is led to the end hard portion 308 side of the inserting portion 303 through the light guide fiber bundle, and radiated to the outside from the illumination window.

Furthermore, an observation image which has entered from the observation window is converted into an electric signal by the imaging element at the image forming position of the objective lens. Moreover, the electric signal is input to the video processor through the signal cable and subjected to signal processing, and then a video signal is supplied to a non-illustrated monitor so that an image is displayed in a screen of the monitor.

Additionally, the insertion support tool 304 has a main extension unit (a first transforming portion) 312 and an end side extension unit (a second transforming portion) 313. The main extension unit 312 has a soft bag-shaped external sheath 314 which can be extended in an inserting direction of the inserting portion 303 of the large-intestine endoscope 302, a base unit 315 which is coupled with a base end portion of this external sheath 314, and an end unit 316 which is coupled with an end portion of the external sheath 314.

The base unit 315 has a substantially cylindrical mouthpiece member 317. The inserting portion 303 of the large-intestine endoscope 302 can be inserted into a cylinder of this mouthpiece member 317. A large-diameter portion 317a having the largest diameter is provided on the base end portion side of the mouthpiece member 317. An end cylindrical portion 317b whose diameter is smaller than that of any other part and allows insertion of the inserting portion 303 of the large-intestine endoscope 302 is provided on the end portion side of the mouthpiece member 317. Further, an intermediate step portion 317c is formed between the large-diameter portion 317a and the end cylindrical portion 317b of the mouthpiece member 317.

Furthermore, the external sheath 314 has an outer sheath 318 and an inner sheath 319. A rear side fixing ring 318a which is fixed at the intermediate step portion 317c of the mouthpiece member 317 is formed at a base end portion of the outer sheath 318. Moreover, an end side of the outer sheath 318 is extended toward the end unit 316 side. Additionally, a folded first bellows portion 318b is formed on this extended portion side.

A folded second bellows portion 319a is formed in the inner sheath 319. This second bellows portion 319a is arranged on the end cylindrical portion 317b of the mouthpiece member 317. Further, a rear side fixing ring 319b which is fixed at a base end portion of the end cylindrical portion 317b is formed at a base end portion of this second bellows portion 319a.

The end unit 316 has an external sheath fixing member 320. The external sheath fixing member 320 has an inner cylindrical portion 320a whose diameter is substantially the same as that of the end cylindrical portion 317b of the mouthpiece member 317 and an outer cylindrical portion 320b whose diameter is larger than that of this inner cylindrical portion 320a. A bent portion 320c which is inwardly bent is formed at an end portion of the outer cylindrical portion 320b. An inner end portion of this bent portion 320c is fixed on an outer peripheral surface of the inner cylindrical portion 320a.

An annular flange portion 324 having a large diameter is provided to protrude from the outer peripheral surface of the inner cylindrical portion 320a on the base end portion side. Furthermore, an outer sheath accommodation space 325 is formed between the inner cylindrical portion 320a and the outer cylindrical portion 320b in front of the flange portion 324. The first bellows portion 318b of the outer sheath 318 is accommodated in this outer sheath accommodation space 325. A front side fixing ring 318c which is fixed at the end portion of the inner cylindrical portion 320a is formed at the end portion of this first bellows portion 318b. Moreover, a front side fixing ring 319c which is fixed on the outer peripheral surface of the inner cylindrical portion 320a is formed at the end portion of the second bellows portion 319a of the inner sheath 319. This front side fixing ring 319c is arranged on the rear side apart from the flange portion 324 of the inner cylindrical portion 320a.

Additionally, in the mouthpiece member 317, a fluid path 326 through which a working fluid such as pressurized air is supplied is formed between the outer sheath 318 and the inner sheath 319 of the external sheath 314. An inner end portion of this fluid path 326 communicates with a part between the outer sheath 318 and the inner sheath 319 of the mouthpiece member 317. Further, an outer end portion of the fluid path 326 is coupled with an air supply mouthpiece 327 arranged on an outer peripheral surface of the mouthpiece member 317. One end portion of an air supply tube 328 is coupled with this air supply mouthpiece 327. The other end portion of this air supply tube 328 is coupled with non-illustrated air supply means 329 such as an air supply pump. Furthermore, when the external sheath 314 of the insertion support tool 304 is extended, a working fluid such as pressurized air is supplied from this air supply tube 328 to a part between the outer sheath 318 and the inner sheath 319 through the air supply mouthpiece 327 and the fluid path 326, and the external sheath 314 is extended in the inserting direction of the inserting portion 303.

FIG. 50 shows an end side extension unit 313. This end side extension unit 313 has a soft bag-shaped external sheath 341 which can be extended in the inserting direction of the inserting portion 303 of the large-intestine endoscope 302, a substantially cylindrical rear side frame member 342 which is coupled with a base end portion of this external sheath 341, and an end side frame member 343 which is coupled with an end portion of the external sheath 341.

The inserting portion 303 of the large-intestine endoscope 302 can be inserted into a cylinder of the rear side frame member 342. A large-diameter portion 342a is provided on a base end portion side of the rear side frame member 342. An end cylindrical portion 342b whose diameter is smaller than that of any other part and allows insertion of the inserting portion 303 of the large-intestine endoscope 302 is provided on an end portion side of the rear side frame member 342.

Moreover, the external sheath 341 has an outer sheath 344 and an inner sheath 345. A rear side fixing ring 344a which is fixed at the large-diameter portion 342a of the rear side frame member 342 is formed at a base end portion of the outer sheath 344. Additionally, an end side of the outer sheath 344 is extended toward the end side frame member 343. Further, a folded first bellows portion 344b is formed on this extended portion side.

A folded second bellows portion 345a is formed in the inner sheath 345. This second bellows portion 345a is arranged on the end cylindrical portion 342b of the rear side frame member 342. Furthermore, a rear side fixing ring 345b which is fixed at the base end portion of the end cylindrical portion 342b is formed at a base end portion of this second bellows portion 345a.

An annular flange portion 347 having a large diameter is provided to protrude from an outer peripheral surface of the end side frame member 343 on the base end portion side. Moreover, an outer sheath accommodation portion 348 is formed on the outer peripheral surface of the end side frame member 343 in front of the flange portion 347. The first bellows portion 344b of the outer sheath 344 is accommodated in this outer sheath accommodating portion 348. A front side fixing ring 344c which is fixed at the end portion of the end side frame member 343 is formed at the end portion of this first bellows portion 344b. Further, in the inner sheath 345, a front side fixing ring 345c which is fixed on the outer peripheral surface of the end side frame member 343 is formed at the end portion of the second bellows portion 345a. This front side fixing ring 345c is arranged on the rear side of the end side frame member 343 apart from the flange portion 347.

Furthermore, a suction path 349 for enabling a suction force between the outer sheath 344 and the inner sheath 345 of the external sheath 341 is formed in the rear side frame member 342. An inner end portion of this suction path 349 communicates with a part between the outer sheath 344 and the inner sheath 345 of the rear side frame member 342. Moreover, an outer end portion of the suction path 349 is coupled with a suction tube 350. This suction tube 350 is coupled with suction means 351 such as a non-illustrated suction pump.

Additionally, when the end side extension unit 313 is not operated, a suction force from the suction tube 350 is exercised between the outer sheath 344 and the inner sheath 345 of the rear side frame member 342 through the suction path 349 so that the end side extension unit 313 is held in the contracted shape. Further, when the end side extension unit 313 is operated, the suction force which is exercised between the outer sheath 344 and the inner sheath 345 of the rear side frame member 342 is interrupted. In this case, the external sheath 341 is extended in the inserting direction of the inserting portion 303 in accordance with an inserting operation of the inserting portion 303 of the large-intestine endoscope 302.

Furthermore, the end side frame member 343 has an endoscope attachment hole 331 at a substantial shaft center portion thereof. A front side of this endoscope attachment hole 331 is sealed by a transparent cover glass 332. Moreover, the end hard portion 308 of the inserting portion 303 of the large-intestine endoscope 302 is coupled with the endoscope attachment hole 331 of the end side frame member 343 in a fitting state.

Additionally, a soft coupling tube 352 is installed between the main extension unit 312 and the end side extension unit 313. This coupling tube 352 is arranged at a position corresponding to the curving portion 307 of the large-intestine endoscope 302. Therefore, the coupling tube 322 can freely transform in accordance with a curving operation of the curving portion 307 of the large-intestine endoscope 302.

A function of the above-mentioned configuration will now be described. At the time of an operation of inserting the inserting portion 303 of the large-intestine endoscope 302 into the large intestine (a rectum) H2 from the anus H1 of a patient H, the endoscope insertion apparatus 301 according to this embodiment is used in a state where the insertion support tool 304 is previously attached to the inserting portion 303 of the large-intestine endoscope 302 before insertion. In a state before inserting the inserting portion 303 of the large-intestine endoscope 302 into the insertion support tool 304, the insertion support tool 304 is held with the external sheath 314 of the main extension unit 312 and the external sheath 341 of the end side extension unit 313 being respectively contracted. In this state, an operation of attaching the insertion support tool 304 with respect to the inserting portion 303 of the large-intestine endoscope 302 (an insertion support tool attachment step) is carried out.

At the time of this insertion support tool attachment operation, the inserting portion 303 of the large-intestine endoscope 302 is inserted into the cylinder of the mouthpiece member 317 from the end side through the opening portion 317e on the base end surface of the mouthpiece member 317 of the insertion support tool 304. Further, as shown in FIG. 50, the end hard portion 308 of the inserting portion 303 is coupled with the endoscope attachment hole 331 of the end side frame member 343 in the fitting state so that an assembled unit 333 in which the insertion support tool 304 is assembled at the end portion of the inserting portion 303 of the large-intestine endoscope 302 is set.

After attachment of the insertion support tool 304, the assembled unit 333 is inserted into the large intestine (the rectum) H2 from the anus H1 of the patient H (a first insertion step). At the time of this inserting operation, the insertion support tool 304 is inserted into the anus H1 of the patient H from the end side frame member 343 side of the insertion support tool 304. At this time, since the anus H1 of the patient H is usually closed by a sphincter muscle, the operation of pushing the assembled unit 333 into the anus H1 of the patient H while spreading the sphincter muscle of the anus H1.

Furthermore, after the insertion support tool 304 is fixed in the anus H1, a working fluid such as pressurized air is supplied to a part between the outer sheath 318 and the inner sheath 319 of the main extension unit 312 through the air supply mouthpiece 327 and the fluid path 326 from the air supply tube 328. At this time, supply of the working fluid inflates a space between the outer sheath 318 and the inner sheath 319 of the external sheath 314.

Therefore, the entire external sheath 314 is extended in the inserting direction of the inserting portion 303 of the large-intestine endoscope 302. As a result, the insertion support tool 304 moves forward in a state where the end unit 316 is pushed forward with respect to the based unit 315. At this time, the inserting portion 303 of the large-intestine endoscope 302 is moved forward together with the end unit 316 in the inserting direction of this inserting portion 303 (a second insertion step).

Thereafter, when the end portion of the insertion support tool 304 reaches a sharp intestinal canal bent portion in the large intestine H2 of the patient as shown in FIG. 51A, the part of the coupling tube 352 between the main extension unit 312 and the end side extension unit 313 (the part of the curving portion 307 of the large-intestine endoscope 302) is arranged at this position. When the working fluid is further supplied to the external sheath 314 of the main extension unit 312 in this state, the part of the curving portion 307 of the large-intestine endoscope 302 lodges in this sharp intestinal canal bent portion in the large intestine H2 and cannot move forward as shown in FIG. 51B.

Therefore, in such a case, the end side extension unit 313 is activated. When this end side extension unit 313 is activated, driving of the suction means 351 is stopped. In this case, the suction force which is exercised between the outer sheath 344 and the inner sheath 345 of the rear side frame member 342 is interrupted. In this state, the inserting portion 303 of the large-intestine endoscope 302 is pushed in the inserting direction. The external sheath 341 is extended in the inserting direction of the inserting portion 303 in accordance with the pushing operation of this inserting portion 303 as shown in FIG. 51C (a third insertion step). As a result, a part of the curving portion 307 of the large-intestine endoscope 302 is pulled into the end side extension unit 313 and moves over the sharp intestinal canal bent portion in the large intestine H2 of the patient.

Thus, the apparatus having the above-described configuration demonstrates the following effects. That is, in the endoscope insertion apparatus 301 according to this embodiment, attaching the inserting support tool 304 at the inserting portion 303 of the large-intestine endoscope 302 can extend the soft external sheath 314 of the main extension unit 312 of the insertion support tool 304 in the inserting direction of the inserting portion 303 at the time of inserting the inserting portion 303 of the large-intestine endoscope 302, thereby supporting the inserting operation of the inserting portion 303 of the large-intestine endoscope 302. Therefore, it is possible to readily perform the operation of inserting the inserting portion 303 of the large-intestine endoscope 302 into the large intestine (the rectum) H2 of the patient H.

Moreover, in the endoscope insertion apparatus 301 according to this embodiment, the end side extension unit 313 is provided in front of the main extension unit 312. Additionally, when the end portion of the insertion support tool 304 reaches the sharp intestinal canal bent portion in the large intestine H2 of the patient during the operation of inserting the inserting portion 303 of the large-intestine endoscope 302 into the large intestine (the rectum) H2 of the patient H, there may possibly occur a phenomenon that the part of the curving portion 307 of the large-intestine endoscope 302 lodges in this sharp intestinal canal bent portion in the large intestine H2 and cannot move forward as shown in FIG. 51B even if the working fluid is further supplied to the external sheath 314 of the main extension unit 312. In such a case, activating the end side extension unit 313 can extend the external sheath 341 in the inserting direction of the inserting portion 303 as shown in FIG. 51C in accordance with the pushing operation of the inserting portion 303, thereby eliminating the phenomenon that the end portion of the insertion support tool 304 cannot move forward. As a result, it is possible to smoothly and assuredly perform the operation of inserting the assembled unit 333 into the large intestine H2 of the patient H.

It is to be noted that the description has been given as to the example of using the suction means in this embodiment, the suction means may have pressurization means (not shown) for increasing a pressure between the outer sheath 344 and the inner sheath 345. Further, a pressurization control switch (not shown) which is used by an operator to control a timing of pressurization is provided to the pressurization means, and the operator can operate the pressurization control switch at an arbitrary timing to turn on/off pressurization. When the suction means further has the pressurization means, the end side extension unit 313 can be forcibly extended, and the inserting portion 303 is pulled and further inserted into the large intestine by the end side extension unit 313 in accordance with the extending operation of the end side extension unit 313.

According to the above-described configuration, when the operator tries to push the inserting portion 303 forward, it is possible to obtain an effect that the end side of the inserting portion 303 can be further inserted into the large intestine irrespective of the pushing force of the inserting portion 303 under a situation where the pushing force is not transmitted to the end side of the inserting portion 303 and the end side of the inserting portion 303 cannot be moved.

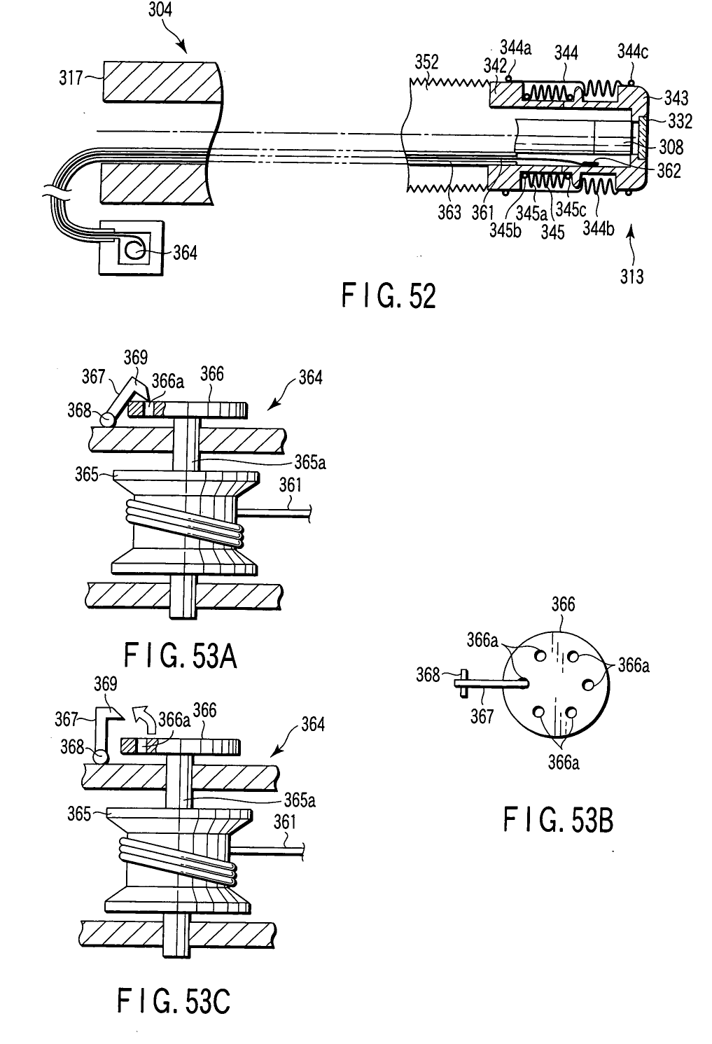

FIGS. 52 to 53C show a 23rd embodiment according to the present invention. This embodiment is obtained by changing the configuration of the insertion support tool 304 of the endoscope insertion apparatus 301 of the 22nd embodiment (see FIGS. 49 to 51C) as follows.

That is, although the 22nd embodiment shows the configuration in which a suction force from the suction tube 350 is utilized as the means for holding the end side extension unit 313 of the insertion support tool 304 in a non-activated state, an operation wire 361 which restricts an advancing operation of an end side frame member 343 is provided in an insertion support tool 304 according to this embodiment. As shown in FIG. 52, a fixed end portion 362 at an end of this operation wire 361 is fixed at a rear end portion of the end side frame member 343.

The operation wire 361 is extended to a mouthpiece member 317 side of the insertion support tool 304 in a state where the operation wire 361 is inserted in a wire guide 363. A fixed end portion 363a at an end of the wire guide 363 is fixed at a rear end portion of a rear side frame member 342.

Moreover, a wire fixing portion 364 is provided to the mouthpiece member 317. As shown in FIG. 53A, this wire fixing portion 364 has a wire take-up roller 365 which takes up the operation wire 361. A rotary disk 366 is fixed at one end portion of a rotary shaft 365a of this wire take-up roller 365. As shown in FIG. 53B, a plurality of locking holes 366a are aligned on this rotary disk 366 along a circumferential direction thereof.

Additionally, a roller fixing lever 367 is provided to the wire fixing portion 364. One end portion of this roller fixing lever 367 is supported to be capable of swiveling around a swiveling shaft 368. A locking claw 369 is provided at the other end portion of the roller fixing lever 367. This locking claw 369 is selectively releasably locked in any locking hole 366a of the rotary disk 366. Further, as shown in FIG. 53A, when the locking claw 369 of the roller fixing lever 367 is locked in any locking hole 366a of the rotary disk 366, rotation of the wire take-up roller 365 is stopped to restrict the advancing operation of the end side frame member 343.

Furthermore, as shown in FIG. 53C, when the roller fixing lever 367 is swiveled to a position at which the locking claw 369 of the roller fixing lever 367 comes off the locking hole 366a of the rotary disk 366, rotation of the wire take-up roller 365 is enabled to move forward the end side frame member 343.

A function of this embodiment having the above-mentioned configuration will now be described. In this embodiment, the end side extension unit 313 of the insertion support tool 304 is switched between a non-activated state and an activated state in accordance with the operation of the roller fixing lever 367 of the wire fixing portion 364. Therefore, this embodiment can obtain the same function and effect as those of the insertion support tool 304 according to the 22nd embodiment.

Moreover, in this embodiment, since the locking claw 369 of the roller fixing lever 367 is locked in any locking hole 366a of the rotary disk 366 when the end side extension unit 313 of the insertion support tool 304 is held in the non-activated state, it is possible to assuredly avoid extension of the external sheath 341 of the end side extension unit 313 when the end side extension unit 313 is in the non-activated state. Therefore, the usability of the end side extension unit 313 of the insertion support tool 304 can be improved.

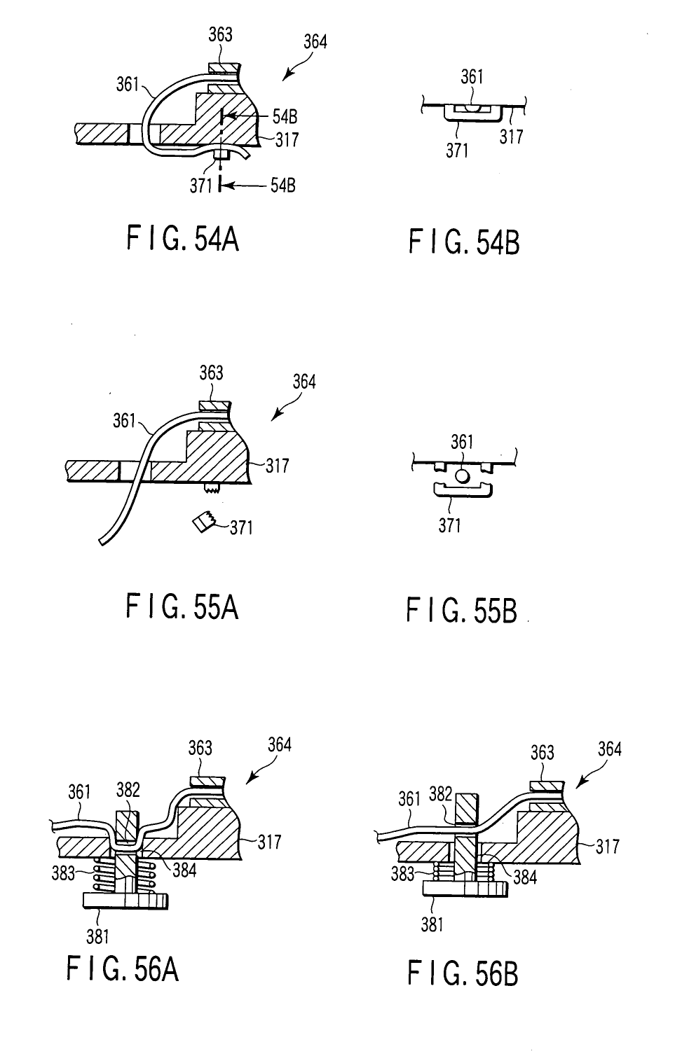

FIGS. 54A to 55B show a first modification of the wire fixing portion 364 assembled with respect to the insertion support tool 304 according to the 23rd embodiment (see FIGS. 52 to 53C). In a wire fixing portion 364 according to this modification, a wire fixing claw 371 which fixes an operation wire 361 is provided to a mouthpiece member 317 as shown in FIGS. 54A and 54B. An advancing operation of an end side frame member 343 is restricted when the operation wire 361 is fixed by this wire fixing claw 371. Further, as shown in FIGS. 55A and 55B, when the wire fixing claw 371 is cut off, the restriction of the operation wire 361 can be released to move forward the end side frame member 343.

In this modification, the advancing operation of the end side frame member 343 is restricted when the operation wire 361 is fixed by the wire fixing claw 371, but cutting off the wire fixing claw 371 can release the restriction of the operation wire 361 to move forward the end side frame member 343. Therefore, this modification can obtain the same function as that of the insertion support tool 304 according to the 22nd embodiment, thereby demonstrating the same effect as that of the insertion support tool 304 according to the 22nd embodiment.

FIGS. 56A and 56B show a second modification of the wire fixing portion 364 assembled with respect to the insertion support tool 304 according to the 23rd embodiment (see FIGS. 52 to 53C). In a wire fixing portion 364 according to this modification, a wire fixing button 381 is provided as shown in FIGS. 56A and 56B. A shaft portion 382 of this wire fixing button 381 is inserted into a through hole 384 formed in a mouthpiece member 317. Moreover, a coil spring 383 which holds the wire fixing button 381 at a fixed position shown in FIG. 56A is provided to the wire fixing portion 364.

Additionally, a wire insertion hole portion 382a into which an operation wire 361 is inserted is formed in the shaft portion 382 of the wire fixing button 381. In a state where the wire fixing button 381 is held at the fixed portion shown in FIG. 56A, this wire insertion hole portion 382a is inserted in the through hole 384 of the mouthpiece member 317. Therefore, the operation wire 361 inserted in the wire insertion hole portion 382a of the shaft portion 382 of the wire fixing button 381 is restricted to a state where the operation wire 361 cannot move, due to the spring force of the coil spring 383. In this state, an advancing operation of an end side frame member 343 is restricted. Further, as shown in FIG. 56B, when the wire fixing button 381 is pushed in against a spring force of the coil spring 383, the wire insertion hole portion 382a of the wire fixing button 381 is pushed out of the through hole 384 of the mouthpiece member 317. As a result, the restriction of the operation wire 361 is released to enable the advancing operation of the end side frame member 343.

In the wire fixing portion 364 according to this modification, the end side extension unit 313 of the insertion support tool 304 is switched between a non-activated state and an activated state by the operation of the wire fixing button 381. Therefore, this embodiment can obtain the same function as that of the insertion support tool 304 according to the 22nd embodiment, and hence this modification can demonstrate the same effect as that of the insertion support tool 304 according to the 22nd embodiment.

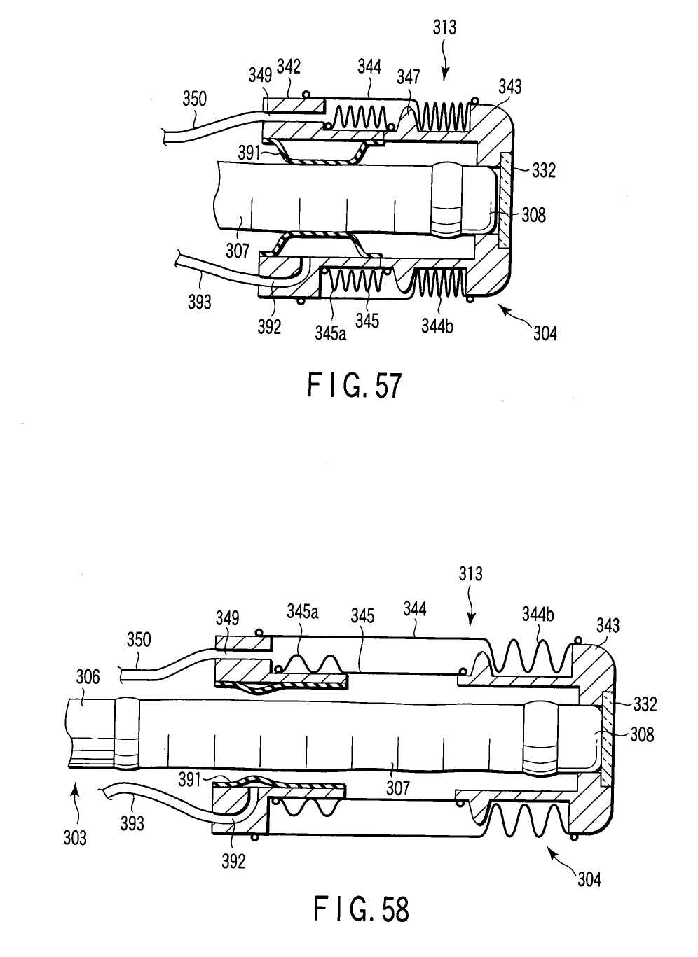

FIGS. 57 and 58 show a 24th embodiment according to the present invention. This embodiment is obtained by changing the configuration of the insertion support tool 304 of the endoscope insertion apparatus 301 according to the 22nd embodiment (see FIGS. 49 to 51C) as follows.

That is, in this embodiment, a balloon 391 is provided as means for holding an end side extension unit 313 of an insertion support tool 304 in a non-activated state. This balloon 391 is fixed on an inner peripheral surface of a rear side frame member 342. Furthermore, an air supply tube line 392 is formed in the rear side frame member 342. An inner end portion of this air supply tube line 392 communicates with the inside of the balloon 391. An outer end portion of the air supply tube line 392 is coupled with one end of an air supply tube 393. The other end of the air supply tube 393 is coupled with an air supply control portion which controls supply/discharge of a working fluid such as air.

Moreover, as shown in FIG. 57, in a state where the balloon 391 is inflated, an inner peripheral surface of the balloon 391 is brought into contact with a curving portion 307 part of a large-intestine endoscope 302 with pressure to restrict an advancing operation of an end side frame member 343. Additionally, as shown in FIG. 58, in a state where the balloon 391 is contracted, the inner peripheral surface of the balloon 391 is moved away from the part of the curving portion 307 of the large-intestine endoscope 302, thereby moving forward the end side frame member 343. As a result, an external sheath 341 of an end side extension unit 313 can be extended in an inserting direction of an inserting portion 303 of the large-intestine endoscope 302.

Thus, the apparatus having the above-described configuration demonstrates the following effects. That is, in this embodiment, the end side extension unit 313 of the insertion support tool 304 is switched between a non-activated state and an activated state by the operation of the balloon 391. Therefore, this embodiment can obtain the same function as that of the insertion support tool 304 according to the 22nd embodiment, and hence this modification can demonstrate the same effect as that of the insertion support tool 304 according to the 22nd embodiment.

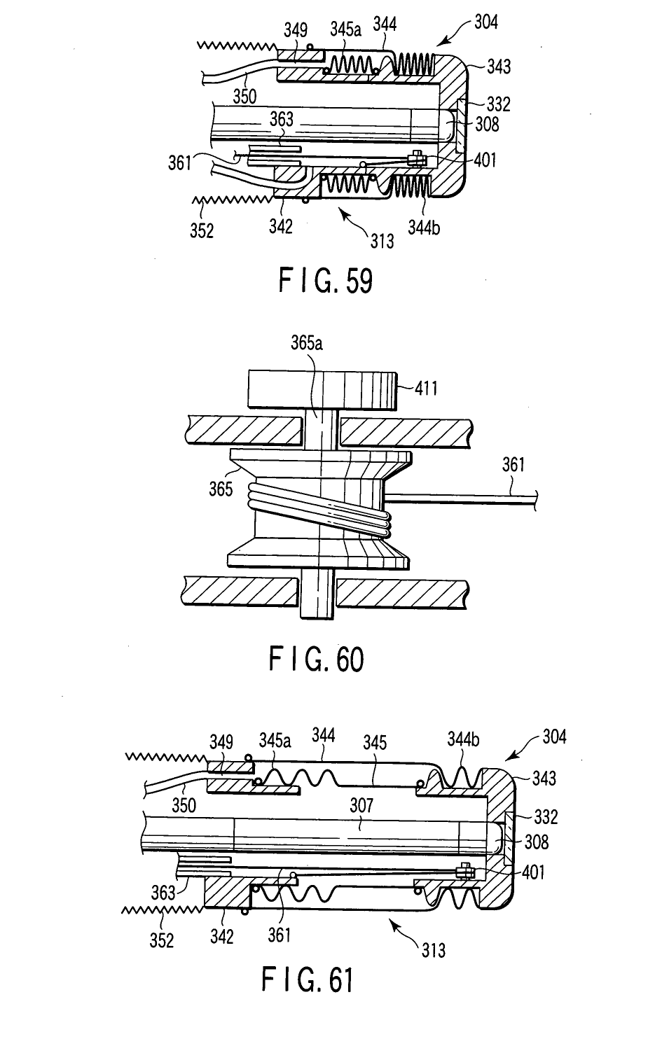

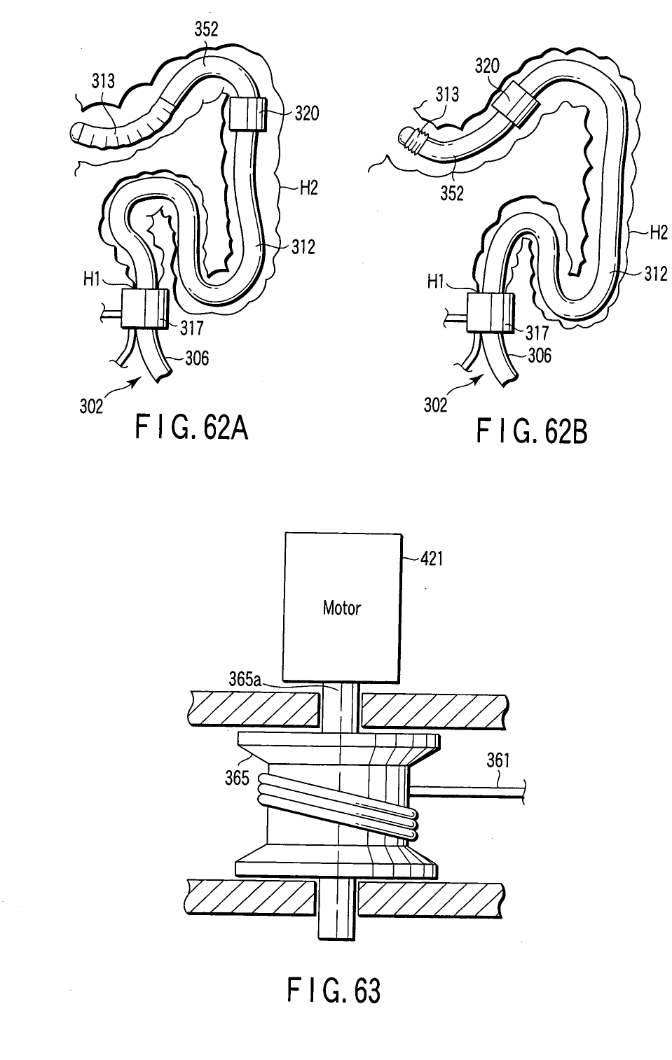

Further, FIGS. 59 to 62B show a 25th embodiment according to the present invention. This embodiment is obtained by changing the configuration of the insertion support tool 304 of the endoscope insertion apparatus 301 according to the 22nd embodiment (see FIGS. 49 to 51C) as follows.

That is, in this embodiment, there is provided an end side extension unit contraction mechanism 400 which uses the operation wire 361 according to the 23rd embodiment (see FIGS. 52 to 53C) to pull an end side extension unit 313 in a contracting direction. In this end side extension unit contraction mechanism 400, a pulley 401 is provided on an inner peripheral surface of an end side frame member 343. An end portion of an operation wire 361 is wound around this pulley 401. The end portion of the operation wire 361 folded back by this pulley 401 is fixed at a front end portion of a rear side frame member 342.

Furthermore, a rotating operation handle 411 is fixed to a mouthpiece member 317 at one end portion of a rotary shaft 365a of a wire take-up roller 365. Operating this rotating operation handle 411 can manipulate the operation wire 361 in either a direction along which the operation wire 361 is taken up by the wire take-up roller 365 or a direction along which the operation wire 361 is unreeled from the wire take-up roller 365. Here, operating the rotating operation handle 411 in the direction along which the operation wire 361 is taken up by the wire take-up roller 365 can pull the end side extension unit 313 in the contracting direction.

Thus, the apparatus having the above-mentioned configuration can demonstrate the following effects. That is, in the endoscope insertion apparatus 301 according to this embodiment, an external sheath 341 of the end side extension unit 313 is extended in an inserting direction of an inserting portion 303 of the large-intestine endoscope 302 to insert the inserting portion 303 of the large-intestine endoscope 302, and then the rotating operation handle 411 is operated in the direction along which the operation wire 361 is taken up by the wire take-up roller 365, thereby contracting the external sheath 341 of the end side extension unit 313 to an original length thereof. Therefore, the end side extension unit 313 can be used many times, as required.

FIG. 63 shows a modification of the driving device of the end side extension unit 313 of the insertion support tool 304 according to the 25th embodiment (see FIGS. 59 to 62B). In this modification, a driving motor 421 is provided in place of the manual rotating operation handle 411. Thus, this modification can obtain the same function as that of the insertion support tool 304 according to the 25th embodiment, and hence this modification can demonstrate the same effect as that of the insertion support tool 304 according to the 25th embodiment.

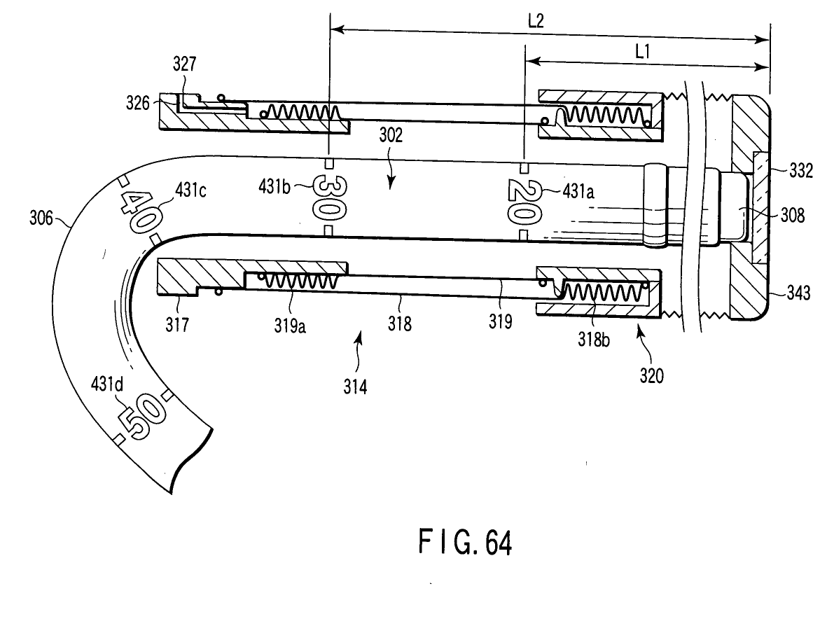

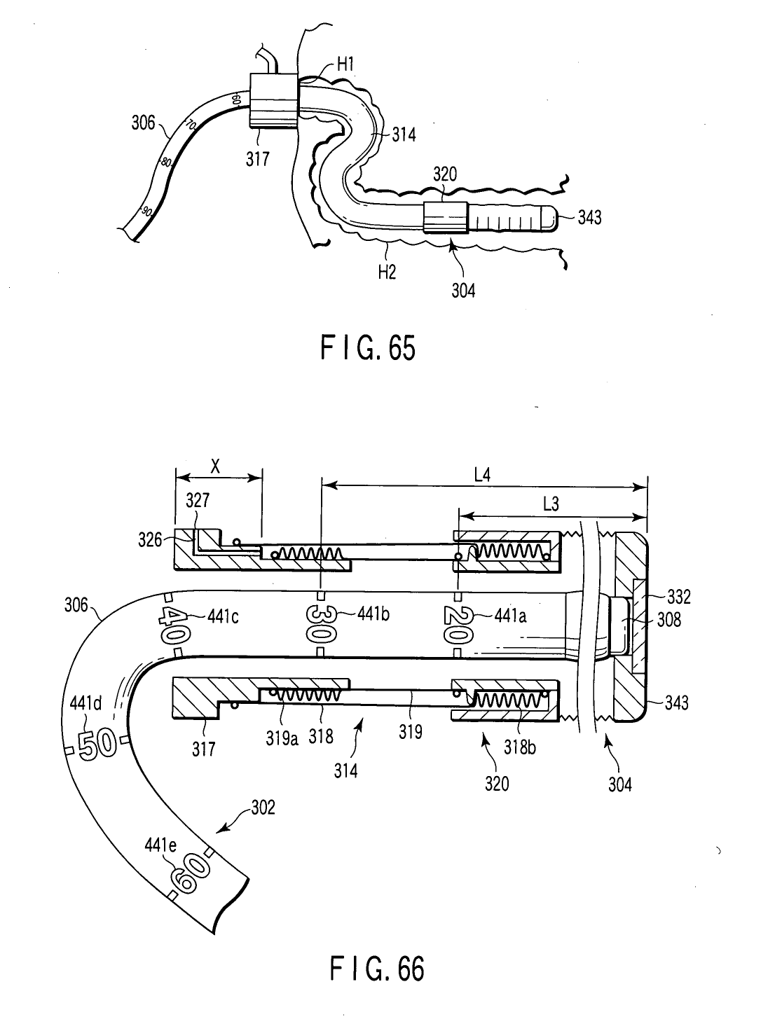

FIGS. 64 and 65 show a 26th embodiment according to the present invention. This embodiment is obtained by changing the configuration of the endoscope insertion apparatus 301 according to the 22nd embodiment (see FIGS. 49 to 51C) as follows.

That is, in this embodiment, a plurality of indices 431a to 431d which indicate a length from an end of an insertion support tool 304 in a state where an inserting portion 303 of a large-intestine endoscope 302 is assembled in the insertion support tool 304 are provided to the inserting portion 303 of the large-intestine endoscope 302 which is assembled in the insertion support tool 304 and used in this state. Here, when a length L1 from the end of the insertion support tool 304 is, e.g., 200 mm, the index 431a having the number “20” is indicated on the inserting portion 303 of the large-intestine endoscope 302. Likewise, when a length L2 from the end of the insertion support tool 304 is, e.g., 300 mm, the index 431b having the number “30” is indicated on the inserting portion 303 of the large-intestine endoscope 302.

Further, in this embodiment, as shown in FIG. 65, a length of the inserting portion 303 of the large-intestine endoscope 302 pulled into the insertion support tool 304 can be confirmed by visually observing the number of each of the indices 431a to 431d indicated on the inserting portion 303 of the large-intestine endoscope 302 at a position of a mouthpiece member 317 of the insertion support tool 304. As a result, an insertion length of the inserting portion 303 of the large-intestine endoscope 302 into a body cavity can be confirmed.

Thus, the apparatus having the above-mentioned configuration demonstrates the following effects. That is, in this embodiment, an insertion length into a body cavity can be confirmed by visually observing the number of each of the indices 431a to 431d indicated on the inserting portion 303 of the large-intestine endoscope 302 at the position of the mouthpiece member 317 of the insertion support tool 304, and hence an end position of the inserting portion 303 of the large-intestine endoscope 302 can be readily grasped. Furthermore, an impossible insertion state can be easily detected.

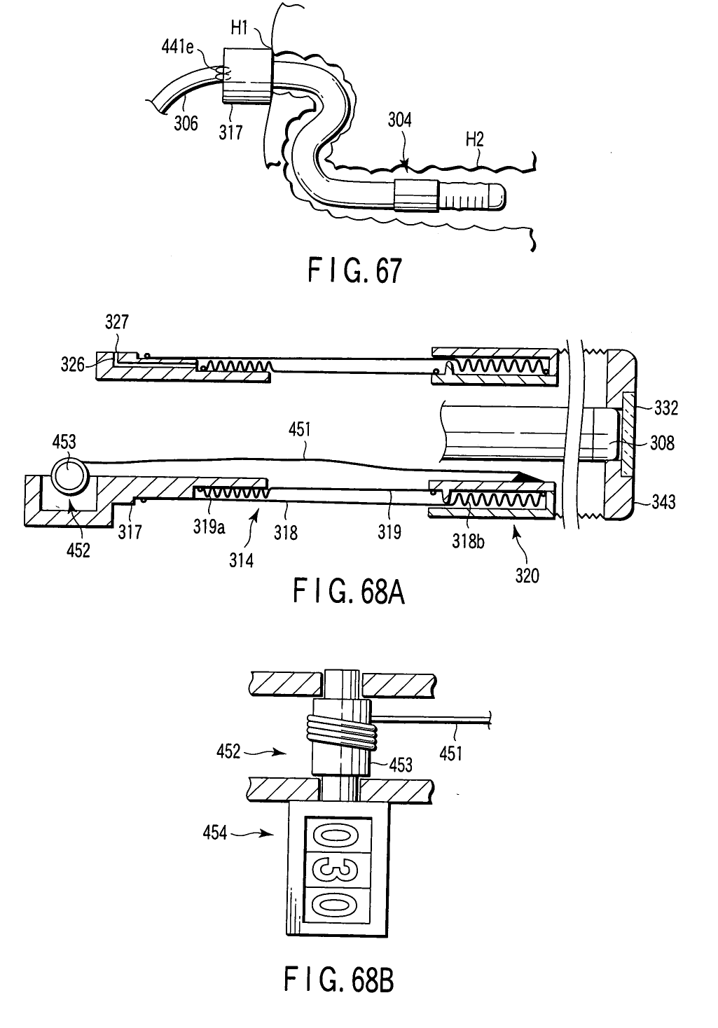

FIGS. 66 and 67 show a 27th embodiment according to the present invention. This embodiment is obtained by changing the configuration of the endoscope insertion apparatus 301 according to the 22nd embodiment (see FIGS. 49 to 51C) as follows.

That is, in this embodiment, a plurality of indices 441a to 441e are provided on an inserting portion 303 of a large-intestine endoscope 302, which is assembled in an insertion support tool 304 and used in this assembled state, at equal intervals from an end of the insertion support tool 304 in a state where the inserting portion 303 of the large-intestine endoscope 302 is assembled in the insertion support tool 304. Here, when a length of a part of the mouthpiece member 317 of the insertion support tool 304 which is exposed to the outside of a body is X, a numeric value obtained by adding a length L3, L4 . . . from the end of the insertion support tool 304 to each of the respective indices 441a to 441e to the length X of the part exposed to the outside of the body is indicated as a part of the respective indices 441a to 441e.

Thus, the apparatus having the above-mentioned configuration demonstrates the following effects. That is, in this embodiment, an insertion length into a body cavity can be further accurately confirmed by visually observing the numeric value of each of the indices 441a to 441d indicated on the inserting portion 303 of the large-intestine endoscope 302 at the position of the mouthpiece member 317 of the insertion support tool 304, and hence an end position of the inserting portion 303 of the large-intestine endoscope 302 can be readily accurately grasped.

FIGS. 68A and 68B show a 28th embodiment according to the present invention. This embodiment is obtained by changing the configuration of the endoscope insertion apparatus 301 according to the 22nd embodiment (see FIGS. 49 to 51C) as follows.

That is, in this embodiment, as shown in FIG. 68A, one end of a measurement wire 451 is fixed to an external sheath fixing member 320 of an end unit 316 of an insertion support tool 304. Further, an insertion length measurement portion 452 is provided to a mouthpiece member 317. A wire take-up roller 453 which takes up the measurement wire 451 is provided to this insertion length measurement portion 452. An insertion length indicator 454 is provided to a rotary shaft of this wire take-up roller 453.

Here, a relationship between the number of revolutions x of the wire take-up roller 453 and an insertion length y is expressed as follows:
y=ax+b (a and b are constants)
Furthermore, the number of revolutions x of the wire take-up roller 453 is detected, and a value of y=ax+b is indicated in the insertion length indicator 454.

Thus, the apparatus having the above-described configuration demonstrates the following effects. That is, in this embodiment, since a value of the insertion length y is indicated in the insertion length indicator 454, the value of the insertion length y can be clearly specified.

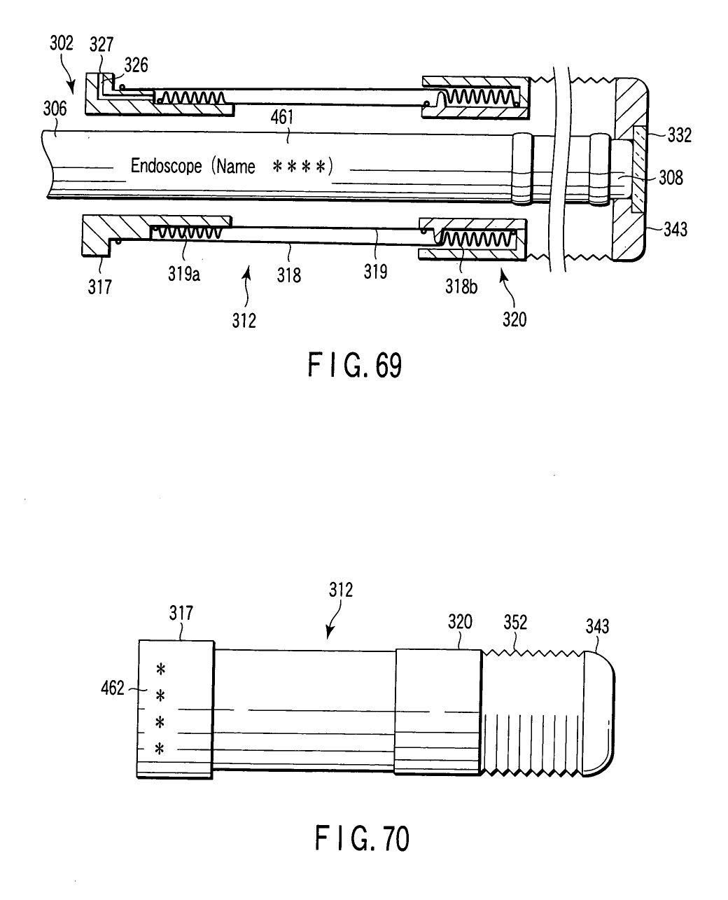

FIGS. 69 and 70 show a 29th embodiment according to the present invention. This embodiment is obtained by changing the configuration of the endoscope insertion apparatus 301 according to the 22nd embodiment (see FIGS. 49 to 51C) as follows.

That is, in this embodiment, as shown in FIG. 69, a label 461 which indicates a model name of a large-intestine endoscope 302 which is combined with an endoscope insertion support tool 304 is provided on the large-intestine endoscope 302. Additionally, as shown in FIG. 70, a label 462 which indicates a model name of the large-intestine endoscope 302 to be combined is provided on the insertion support tool 304.

Thus, the apparatus having the above-mentioned configuration demonstrates the following effects. That is, in this embodiment, even if the endoscope insertion support tool 304 is prepared with a specification which differs in accordance with each model of the endoscope 302 based on a thickness or the like of an inserting portion 303 of the endoscope 302, a wrong combination can be avoided. Therefore, it is possible to prevent an extension defect or a device damage due to a wrong combination of the endoscope insertion support tool 304 and the endoscope 302.

Further, the present invention is not restricted to the foregoing embodiments, and can be of course modified and carried out in many ways without departing from the scope of the invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general invention concept as defined by the appended claims and their equivalents.

What is claimed is:

1. An insertion support tool for a medical instrument, comprising:
a transforming portion which is extendable and contractible along an inserting direction of a flexible inserting portion of the medical instrument into a lumen; and
a cylindrical cover member which is held in a state where it covers the transforming portion when the transforming portion is contracted.

2. The insertion support tool for a medical instrument according to claim 1, wherein the cover member is formed of a material which can prevent an external force applied to a surface of the cover member from being exercised with respect to the transforming portion.

3. The insertion support tool for a medical instrument according to claim 1, wherein the transforming portion has a bag-shaped sheath which is arranged to cover at least a part of a side surface of the inserting portion in the inserting direction and which is extendable in the inserting direction of the inserting portion by supply of a fluid.

4. The insertion support tool for a medical instrument according to claim 1, further having a holding member which holds the state where the cover member covers the transforming portion when the transforming portion is contracted.

5. The insertion support tool for a medical instrument according to claim 4, wherein the holding member has a packing material which packs the insertion support tool.

6. The insertion support tool for a medical instrument according to claim 1, further comprising a protection member including a fixing portion to which at least a part of the insertion support tool is fixed,
wherein the protection member is insertable into and fixable in the lumen prior to insertion of the medical instrument into the lumen.

7. An insertion support tool for a medical instrument, which is detachably attached to the medical instrument including a flexible shaft-shaped inserting portion which is inserted into a lumen, the insertion support tool comprising:
a bag-shaped transforming portion which is arranged outside the inserting portion and which is extendable in an inserting direction of the inserting portion by supply of a fluid;
a base unit having a first transforming portion fixing portion to which a base end portion of the transforming portion is fixed;
an end unit having a second transforming portion fixing portion to which an end portion of the transforming portion is fixed; and
a cylindrical cover member which is arranged on an outer side of the transforming portion, whose base end portion is fixed to the base unit, and which is held in a state where it covers the transforming portion when the transforming portion is contracted.

8. The insertion support tool for a medical instrument according to claim 7, wherein the transforming portion has an outer sheath having a folded first bellows portion and an inner sheath which is arranged on an inner side of the outer sheath and has a folded second bellows portion, and
the base unit has a fluid path through which a working fluid is supplied between the outer sheath and the inner sheath.

9. The insertion support tool for a medical instrument according to claim 7, wherein the end unit has an engagement member which is releasably engaged with an end portion of the cover member and holds a state where the cover member covers the transforming portion when the transforming portion is contracted.

10. The insertion support tool for a medical instrument according to claim 9, wherein the engagement member has a tape which locks a part between the end unit and the cover member.

11. The insertion support tool for a medical instrument according to claim 9, wherein the engagement member has a fixed thread having one end fixed to the end unit and the other end fixed to the base unit.

12. The insertion support tool for a medical instrument according to claim 9, wherein the engagement member has a concavoconvex fitting portion formed in a joint portion of the end unit and the cover member.

13. An insertion method for a medical instrument, comprising:
a step of attaching an insertion support tool to the medical instrument including a shaft-shaped inserting portion which is inserted into a lumen;
a first insertion step of inserting the insertion support tool into the lumen together with the medical instrument while maintaining a state where the transforming portion which is extendable and contractible in an inserting direction of the inserting portion is covered with a cylindrical cover member after attaching the insertion support tool; and
a second insertion step of fixing a base end portion of the insertion support tool in an anus part, then extending the transforming portion in the inserting direction of the inserting portion to protrude from the cover member so that the medical instrument is moved forward in the inserting direction of the inserting portion.

14. An insertion support tool for a medical instrument, having;
a first extension unit which is extendable and contractible in an inserting direction of a flexible inserting portion of the medical instrument into a lumen;
a second extension unit which is extendable and contractible in the inserting direction separately from the first extension unit; and
a coupling member which couples the first extension unit with the second extension unit.

15. The insertion support tool for a medical instrument according to claim 14, wherein the second extension unit is arranged at an end portion of the first extension unit.

16. The insertion support tool for a medical instrument according to claim 14, wherein the first extension unit comprises:
a bag-shaped first transforming portion which is arranged to cover at least a part of a side surface of the inserting portion in the inserting direction and which is extendable in the inserting direction of the inserting portion by supply of a fluid;
a base unit having a first transforming portion fixing portion to which a base end portion of the first transforming portion is fixed;
an end unit having a second transforming portion fixing portion to which an end portion of the first transforming portion is fixed; and
a cylindrical cover member whose end portion is fixed to the end unit and which is held in a state where it covers the first transforming portion when the first transforming portion is contracted, and
the second extension unit has:
a bag-shaped second transforming portion which is arranged to cover at least a part of the side surface of the inserting portion in the inserting direction and which is extendable in the inserting direction of the inserting portion by supply of a fluid;
a cylindrical rear side frame member which is coupled with a base end portion of the second transforming portion; and
an end side frame member which is coupled with an end portion of the second transforming portion,
the coupling member having a coupling tube whose end is fixed to the rear side frame member and whose base end is fixed to the end unit.

17. The insertion support tool for a medical instrument according to claim 16, wherein the first transforming portion has a first outer sheath having a folded first bellows portion and a first inner sheath which is arranged on an inner side of the first outer sheath and has a folded second bellows portion,
the base unit has a first fluid path through which a working fluid is supplied between the first outer sheath and the first inner sheath,
the second transforming portion has a second outer sheath having a folded third bellows portion and a second inner sheath which is arranged on an inner side of the second outer sheath and has a folded fourth bellows portion, and
the rear side frame member has a second fluid path through which the working fluid is supplied between the second outer sheath and the second inner sheath.

18. The insertion support tool for a medical instrument according to claim 14, further having an indicating portion which indicates identification data of the medical instrument to be combined.

19. An insertion method for a medical instrument, comprising:
a step of attaching an insertion support tool to a medical instrument including a flexible shaft-shaped inserting portion which is inserted into a lumen;
a first insertion step of inserting the insertion support tool together with the medical instrument into the lumen after attaching the insertion support tool;
a second insertion step of fixing a base end portion of the insertion support tool in an anus part, and then extending the first transforming portion in an inserting direction of the inserting portion to move forward the medical instrument in the inserting direction of the inserting portion; and
a third insertion step of extending the second transforming portion in the inserting direction of the inserting portion to move forward the medical instrument in the inserting direction of the inserting portion separately from the forward movement operation of the medical instrument by the first transforming portion.
